(19) United States
(12) Patent Application Publication
(10) Pub. No.: US 2025/0059581 A1
(43) Pub. Date: Feb. 20, 2025

(54) METHOD OF BACTERIAL IDENTIFICATION AND TESTING OF BACTERIUM RESISTANT TO ONE OR MORE ANTIBIOTICS

(71) Applicant: Nanyang Technological University, Singapore (SG)

(72) Inventors: Hongwei DUAN, Singapore (SG); Qirong XIONG, Singapore (SG)

(21) Appl. No.: 18/722,396

(22) PCT Filed: Dec. 29, 2022
(86) PCT No.: PCT/SG2022/050942

§ 371 Date: Jun. 20, 2024

(30) Foreign Application Priority Data
Dec. 29, 2021 (SG) 10202114470Q

(51) Int. Cl. C12Q 1/04

(52) U.S. Cl. CPC C12Q 1/04

(57) ABSTRACT

Disclosed herein is a method of detecting an antimicrobial susceptibility of a bacterium to one or more antibiotics, the method requiring the steps of: (a) providing a bacterial mixture that includes a bacterial population including a bacterial species suspected to be resistant to one or more antibiotics, an antibiotic, a metabolic precursor suitable for incorporation into a bacterium, which metabolic precursor is labelled or is capable of being labelled with a detectable moiety, and a nutrient suspension; and (b) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium.

### FIELD OF INVENTION

The current invention relates to a method of detecting a bacterium resistant to one or more antibiotics and a method of detecting an antimicrobial susceptibility of a bacterium to one or more antibiotics.

### BACKGROUND

The listing or discussion of a prior-published document in this specification should not necessarily be taken as an acknowledgement that the document is part of the state of the art or is common general knowledge.

Antibiotics have greatly reduced illness and deaths resulting from infectious diseases since their introduction into medicine in the 1940s. However, conventional antibiotics used widely in medicine, agriculture, and veterinary industry are becoming increasingly ineffective in the face of antimicrobial resistance (AMR) bacteria, especially those belonging to the ESKAPE group (Enterococcus faecium, Staphylococcus aureus, Klebsiella pneumoniae, Acinetobacter baumannii, Pseudomonas aeruginosa, and Enterobacter species). The overuse or misuse of antibiotics and the slow development of new antibiotics in the past decades are making the situation even more challenging. The Centers for Disease Control and Prevention (CDC) of the USA estimated that antibiotic resistance is responsible for more than 2 million infections and 23,000 deaths each year in the USA. In the European Union, the drug-resistant pathogens kill at least 25,000 infected people annually. In Singapore hospitals, a significant 35-50% of bacterial infections are now resistant to front-line antibiotics.

Early detection of resistant bacteria and timely identification of suitable antibiotics are critical for improving patient care, pinpointing the source of infection, and preventing the spread of resistance. Typically, matching an unknown infection to an antibiotic is done by culturing bacteria for identification, followed by exposing the microbes to different antibiotics and observing the best working therapy (i.e. culture-dependent broth dilution and disk diffusion method). Results from these susceptibility tests usually take 24-48 hours, and some cases can take weeks. Such extensive waiting time is challenging for choosing appropriate antibiotics and leaves gaps for resistance development. Genetic detection methods, such as quantitative real-time PCR (qPCR) and fluorescence in situ hybridization (FISH), have shown to be particularly promising as highly sensitive tools for the detection of specific antibiotic resistance genes in bacteria. The correlation between genotypes and phenotypic resistance, however, may not be straightforward. When the level of expression was low, the presence of resistance genes might not be always indicative of a resistant bacterium. It is also noteworthy that genetic detection is only applicable to existing resistance genes and not to their mutations-carrying variants. Recently, there have been efforts in developing faster bacterial resistance assay technologies by characterizing the physical features of bacterial growth (e.g., the number, size, and length of bacterial cells). However, the accuracy and reliability of these approaches require further improvement and validation.

Metabolic labeling refers to methods in which the biosynthesis machinery is used to incorporate detection or affinity tags into the target biomolecules of living cells. Typically, this is accomplished by culturing cells in media in which a specific natural molecular building block (e.g. lipid, amino acid, nucleotide, and sugar) has been replaced with a tagged chemical analog. Cells use the chemical analog instead of the natural biomolecule to synthesize or modify lipid, proteins, glycan, nucleic acids, etc. Advances in bioorthogonal chemistry using catalyst-free click reactions (i.e. azide/strained cyclooctyne and trans-cyclooctene/tetrazine reactions) (Sletten, E. M. et al., Angew. Chem. Int. Ed 2009, 48, 6974-6998, and Scinto, S. L. et al., Nat. Rev. Methods Primers 2021, 1:30, 1-23) has imparted efficient metabolic labeling of biomolecules in living systems. Despite the considerable progress in metabolic labeling techniques for mammalian cells, their application in bacteria is not as widely explored.

Therefore, there exists a pressing need for broadly applicable methods to enable rapid, sensitive, and high-throughput measurement of antibiotic susceptibility.

### SUMMARY OF INVENTION

1. A method of detecting an antimicrobial susceptibility of a bacterium to one or more antibiotics, the method comprising the steps of:
(a) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor is labelled or is capable of being labelled with a detectable moiety; and
   a nutrient suspension; and
(b) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and:
   when the bacterial mixture of step (a) comprises a metabolic precursor that is capable of being labelled with a detectable moiety, then a detectable moiety suitable to conjugate to the metabolic precursor is added to the antibiotic-challenged bacterial mixture.

2. The method according to Clause 1, wherein the method comprises further steps of:
(c) binding an antibody and/or an aptamer to a resistant bacterium, if present, in the antibiotic-challenged bacterial mixture; and
(d) detecting the presence or absence of the resistant bacterium.

3. The method according to Clause 2, wherein the binding of the antibody and/or the aptamer allows for the separation of the resistant bacterium, if present, from the antibiotic-challenged bacterial mixture.

4. The method according to Clause 2 or Clause 3, further comprising a step of increasing a concentration of the resistant bacterium, if present, before conducting a detection step.

5. The method according to any one of the preceding clauses, wherein the metabolic precursor suitable for incorporation into a bacterium is incorporated into a bacterial cell wall.

6. The method according to any one of the preceding clauses, wherein the metabolic precursor suitable for incorporation into a bacterium is one that is capable of being labelled with a detectable moiety and is selected from one or more of the group consisting of lipid II, and, more particularly, a D-amino acid, 3-deoxy-D-manno-octulosonic acid (KDO), trehalose, fucose, and N-acetyl glucosamine, where each of the lipid II, D-amino acid, 3-deoxy-D-manno-octulosonic acid (KDO), trehalose, fucose, and N-acetyl glucosamine bears a click handle suitable to form one or more covalent bonds to a detectable moiety.

7. The method according to Clause 6, wherein the detectable moiety is selected from one or more of the group consisting of a fluorescent dye, an enzyme, and a protein binding moiety (e.g. biotin), wherein each of the fluorescent dye, enzyme, and protein binding moiety (e.g. biotin) bears a click handle suitable to form one or more covalent bonds to the metabolic precursor suitable for incorporation into a bacterium that is capable of being labelled with a detectable moiety.

8. The method according to:
Clause 6, wherein the click handle on the metabolic precursor is selected from one or more of the group consisting of azide, alkyne, trans-cycloctene (TCO), tetrazine, dibenzocyclooctyne (DBCO), and thiol; and
Clause 7, wherein the click handle on the detectable moiety is selected from one or more of the group consisting of azide, alkyne, trans-cycloctene (TCO), tetrazine, dibenzocyclooctyne (DBCO), and thiol, provided that
the click handle on the metabolic precursor suitable for incorporation into a bacterium that is capable of being labelled with a detectable moiety complements the click handle on the detectable moiety.

9. The method according to any one of Clauses 1 to 5, wherein the metabolic precursor suitable for incorporation into a bacterium that is labelled with a detectable moiety is selected from:
one or more of the group consisting of lipid II, and, more particularly, a D-amino acid, 3-deoxy-D-manno-octulosonic acid (KDO), trehalose, fucose, and N-acetyl glucosamine; conjugated to
one or more of the group consisting of a fluorescent dye, an enzyme, and a protein binding moiety (e.g. biotin).

10. The method according to any one of Clauses 2 and 3 to 9, as dependent upon Clause 2, wherein the antibody and/or the aptamer is an antibody.

11. The method according to any one of Clauses 2 and 3 to 10, as dependent upon Clause 2, wherein the step of detecting the presence or absence of the resistant bacterium involves one or more of fluorescent, colourimetric, surface-enhanced Raman spectroscopy (SERS), chemiluminescence, and electrochemical detection.

12. The method according to any one of Clauses 2 and 3 to 11, as dependent upon Clause 2, wherein the method is a metabolic and immune co-labelling fluorescent assay, a microarray assay, an enzyme-linked immunosorbent assay (ELISA), a lateral flow assay, a chemiluminescence assay, an electrochemical assay, and a surface-enhanced Raman spectroscopy (SERS)-dependent microfluidic assay.

13. The method according to any one of Clauses 2 and 3 to 12, as dependent upon Clause 2, wherein the method comprises the steps of:
(i) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor is labelled or is capable of being labelled with a detectable moiety; and
   a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
   when the bacterial mixture of step (i) comprises a metabolic precursor that is capable of being labelled with a detectable moiety, then a detectable moiety suitable to conjugate to the metabolic precursor is added to the antibiotic-challenged bacterial mixture prior to conducting step (iii);
(iii) placing a portion of the antibiotic-challenged bacterial mixture into a well comprising an antibody specific towards the bacterial species suspected to be resistant to one or more antibiotics to capture the bacterial species to provide a captured sample; and
(iv) providing a stimulus to the captured sample and detecting a signal in response to the stimulus, where a response indicates a bacterial species that is resistant to one or more antibiotics.

14. The method according to any one of Clauses 2 and 3 to 12, as dependent upon Clause 2, wherein the method comprises the steps of:
(i) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor is labelled or is capable of being labelled with a detectable moiety; and
   a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
   when the bacterial mixture of step (i) comprises a metabolic precursor that is capable of being labelled with a detectable moiety, then a detectable moiety suitable to conjugate to the metabolic precursor is added to the antibiotic-challenged bacterial mixture prior to conducting step (iii);
(iii) contacting the antibiotic-challenged bacterial mixture with an antibody specific towards the bacterial species suspected to be resistant to one or more antibiotics to provide a bacteria-antibody conjugate mixture;
(iv) contacting the bacteria-antibody conjugate mixture with a labelled protein comprising a second detectable moiety that is capable of conjugating to the antibody to provide a metabolic-immune co-labelled conjugate mixture; and
(v) providing one or more stimuli to the metabolic-immune co-labelled conjugate mixture, where detection of a signal from both the first and second detectable moieties in response to the stimulus indicates a bacterial species that is resistant to one or more antibiotics.

15. The method according to any one of Clauses 2 and 3 to 12, as dependent upon Clause 2, wherein the method comprises the steps of:
(i) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor comprises a protein binding moiety that can be detected or is capable of being conjugated to a protein binding moiety that can be detected; and
   a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
   when the bacterial mixture of step (i) comprises a metabolic precursor that is capable of being conjugated to a protein binding moiety, the protein binding moiety suitable to conjugate to the metabolic precursor is added to the antibiotic-challenged bacterial mixture prior to conducting step (iii);
(iii) breaking up the bacteria in the bacterial mixture with a suitable material to provide a bacterial fragment mixture;
(iv) placing a portion of the bacterial fragment mixture into a well comprising an antibody specific towards the bacterial species suspected to be resistant to one or more antibiotics to capture fragments of the bacterial species to provide a captured sample;
(v) providing a protein suitable to bind to the protein binding moiety to the captured sample, which protein is conjugated to an enzyme capable of producing a detectible signal when supplied with a suitable substrate, to provide a capture sample ready for detection; and
(vi) providing a suitable substrate to the capture sample ready for detection, where the presence of the substrate generates one or more of a chemiluminescence, an electrochemical and, more particularly, a colourimetric response, which response indicates a bacterial species that is resistant to one or more antibiotics.

16. The method according to Clause 15, wherein the protein suitable to bind to the protein binding moiety is streptavidin conjugated to horse radish peroxidase.

17. The method according to any one of Clauses 2 and 3 to 12, as dependent upon Clause 2, wherein the method comprises the steps of:
(i) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor comprises a protein binding moiety that can be detected or is capable of being conjugated to a protein binding moiety that can be detected; and
   a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
   when the bacterial mixture of step (i) comprises a metabolic precursor that is capable of being conjugated to a protein binding moiety, the protein binding moiety suitable to conjugate to the metabolic precursor is added to the antibiotic-challenged bacterial mixture prior to conducting step (iii);
(iii) breaking up the bacteria in the bacterial mixture with a suitable material to provide a bacterial fragment mixture;
(iv) providing a strip cassette comprising:
   a sample well comprising a conjugate pad and a sample pad in fluid communication with each other, the conjugate pad comprising a protein conjugated to a metal nanoparticle; and
   a membrane in fluid communication with the sample well, the membrane comprising a control line comprising an antibody suitable to capture a protein conjugated to a coloured microsphere or a metal nanoparticle and a test line comprising an antibody specific towards the bacterial species suspected to be resistant to one or more antibiotics to capture fragments of the bacterial species; and
(v) adding the bacterial fragment mixture to the sample well, where the bacterial fragment mixture mixes with the protein conjugated to a coloured microsphere or a metal nanoparticle in the conjugate pad to provide a coloured microsphere conjugate mixture or a metal nanoparticle conjugate mixture and the coloured microsphere conjugate mixture or the metal nanoparticle conjugate mixture passes through the test line and the control line, whereupon the visual detection of the control line and test line indicates a bacterial species that is resistant to one or more antibiotics.

18. The method according to any one of Clauses 2 and 3 to 12, as dependent upon Clause 2, wherein the method comprises the steps of:
(i) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor comprises a protein binding moiety that can be detected or is capable of being conjugated to a protein binding moiety that can be detected; and a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
   when the bacterial mixture of step (i) comprises a metabolic precursor that is capable of being conjugated to a protein binding moiety, the protein binding moiety suitable to conjugate to the metabolic precursor is added to the antibiotic-challenged bacterial mixture prior to conducting step (iii);
(iii) breaking up the bacteria in the bacterial mixture with a suitable material to provide a bacterial fragment mixture;
(iv) adding a plurality of magnetic chain structures to the bacterial fragment mixture to provide a Magchain bound bacterial fragment mixture, where the magnetic chain structures comprise a chain of magnetic particles having a polydopamine coating and an antibody specific towards the bacterial species suspected to be resistant to one or more antibiotics to capture fragments of the bacterial species;
(v) introducing the Magchain bound bacterial fragment mixture into a sample well of a microfluidic device and supplying a surface-enhanced Raman spectroscopy (SERS) probe bound to streptavidin to the sample well to form a magnetic chain-bacterial fragment-SERS probe sandwich complex in the sample well;
(vi) manipulating the magnetic chain-bacterial fragment-SERS probe sandwich complexes through the microfluidic device to a concentration and detection well, followed by washing; and
(vii) seeking to detect a Raman spectroscopic signal for the magnetic chain-bacterial fragment-SERS probe sandwich complex, where detection of the signal indicates a bacterial species that is resistant to one or more antibiotics.

19. The method according to any one of Clauses 15 to 18, wherein the protein binding moiety is biotin.

### DRAWINGS

FIG. 1 depicts the concept of metabolic-immune labelling (MILab) for integrated bacterial identification and antimicrobial susceptibility testing.

FIG. 2 depicts (a) schematic illustration of metabolic labeling of S. aureus by incorporation of unnatural D-amino acids into peptidoglycan (PG) peptide stems and click reaction with DBCO-fluorophore for subsequent fluorescence imaging, (b) laser confocal scanning microscopy (LCSM) images of S. aureus using D-Ala-N₃ as metabolic substrate followed by reaction with DBCO-AF488 (green) for labelling. 4′,6-diamidino-2-phenylindole (DAPI; blue) was used for nuclear staining, (c) confocal fluorescence images showing the selectively metabolic labelling of drug-resistant S. aureus (MRSA BAA39) under penicillin G treatment, (d) flow cytometry showing the labelling degree of individual bacteria when treated by varying concentration of antibiotics, (e) fluorescence intensity of susceptible and resistant S. aureus over penicillin G (16 μg/mL) treatment from 0.5 to 5 h, and (f) fluorescence intensity of susceptible and resistant S. aureus with different antibiotic concentration (2-128 μg/mL).

FIG. 3 depicts (a) the confocal fluorescent images of metabolic labelling of S. aureus 29213 and MRSA BAA39 with varying D-Ala-N₃ concentration, (b) mean fluorescence intensity (MFI) of S. aureus labeling with varying concentration of D-Ala-N₃, and (c) the potential bacterial growth inhibition of D-Ala-N₃ with different concentration. The concentration of 1.5 mM would not have significant inhibition on bacterial growth.

FIG. 4 depicts (a) the confocal fluorescence and bright-field images of S. aureus 29213 with different click reaction time showing relationship between labeling intensity and click reaction time, (b) quantitative analysis of fluorescence intensity of S. aureus by flow cytometry, (c) mean fluorescence intensity of single S. aureus, (d) flow cytometry analysis of azido-incorporated S. aureus reacted with DBCO-AF488 from 0 to 20 μM, and (e) mean fluorescence intensity of metabolically labelled S. aureus.

FIG. 5 depicts (a)-(e) metabolic labelling of S. aureus 29213 and BAA39 in response to a panel of antibiotics with different treatment mechanism, and (f)-(j) standard broth dilution test (OD measurement) of S. aureus 29213 and BAA39 in response to the same antibiotics.

FIG. 6 depicts (a) schematic illustration of metabolic labeling of Gram-negative bacteria, (b) Confocal laser scanning microscopy images showing metabolic labelling of E. coli by using KDO-N₃, and (c) metabolic labelling of E. coli EC 958 (resistant) and E. coli ATCC 8739 (susceptible) under penicillin G treatment with different concentration.

FIG. 7 depicts (a) confocal images showing metabolic labelling of MRSA BAA38, BAA40,E. faecalis, B. subtilis, S. mutans and C. difficile, (b) schematic illustration of metabolic-immune co-labelling of S. aureus, (c) immuno-labelling of S. aureus with or without metabolic labelling, and (d) flow cytometry showing the metabolic-immune labelling of all 4 strains of S. aureus and 4 other Gram-positive bacterial strains.

FIG. 8 depicts (a) schematic illustration of MILab-microarray assay, (b) scanning electron microscopy (SEM) image of captured bacteria on microarray substrate, (c) MILab-microarray assay of S. aureus with and without metabolic labelling, (d) MILab-microarray assay of drug-susceptible and resistant S. aureus against a panel of commonly used antibiotics, (e) MILab-microarray assay of SA 29213 and BAA38 with varying drug treatment time, (f) fluorescence intensity of spots analysed by ImageJ, (g) MILab-microarray assay of SA 29213 and BAA38 with different drug concentration, and (h) fluorescence intensity of spots analysed by ImageJ.

FIG. 9 depicts MILab-microarray assay sensitivity for detection of S. aureus under the optimized condition, including (a) fluorescence microscopy images of MILab-microarray assays, and (b) mean of fluorescence intensity of spots.

FIG. 10 depicts (a) schematic illustration of MILab-enzyme-linked immunosorbent assay (MILab-ELISA) and MILab-lateral flow assay (MILab-LFA), (b) MILab-ELISA for susceptibility screening of S. aureus against a panel of commonly used antibiotics, (c) photo of MILab-ELISA with varying drug treatment time for antibiotic exposure time testing of susceptible and resistant S. aureus, (d) optical density (OD) intensity at 650 nm of MILab-ELISA with varying drug treatment time for antibiotic exposure time testing, (e) photo of MILab-ELISA with different drug concentration for antibiotic dose-dependent testing of susceptible and resistant S. aureus, (f) OD intensity at 650 nm of MILab-ELISA with different drug concentration for antibiotic dose-dependent testing, (g) MILab-LFA for screening of 4 strains of S. aureus against a panel of antibiotics, (h) photo of MILab-LFA with different drug treatment time for antibiotic exposure time, and (i) photo of MILab-LFA with varying drug concentration for dose-dependent testing of susceptible and resistant S. aureus.

FIG. 11 depicts sensitivity of MILab-ELISA for detection of S. aureus under the optimized condition, including (a) photo of MILab-ELISA, and (b) OD650 in response to the bacterial concentration. The limit of detection is 1×10² CFU/mL (CFU: colony forming units).

FIG. 12 depicts sensitivity of MILab-LFA for detection of S. aureus 29213 ranging from 5×10³ to 5×10⁶ CFU/mL.

FIG. 13 depicts (a) schematic illustration of the preparation of functionalized magnetic nanochain (Magchain), (b) SEM image of Magchain, and (c) transmission electron microscopy (TEM) image of Magchain.

FIG. 14 depicts schematic illustration of the preparation of SERS-encoded nanoprobes.

FIG. 15 depicts (a) schematic illustration of MILab-based microfluidic biochip (MiChip) assay, (b) photograph of the 10-channels microfluidic biochip, (c) schematic of a single unit of array for analysis of sample for Raman microscopy measurement, which the dimensions of channels and chambers are labelled, (d) SERS responses to pure culture S. aureus ranging from 5 to 10⁴ CFU/mL (Inset: SERS peak intensity at 1341 cm⁻¹ as a function of logarithmic concentration of S. aureus), (e) SERS signal of susceptible and resistant S. aureus under penicillin G treatment with different incubation time, (f) SERS signal of susceptible and resistant S. aureus under penicillin G treatment with different drug concentration, and (g) heat map of MILab-MiChip assay for high-throughput screening of 4 strains of S. aureus against a panel of antibiotics.

FIG. 16 depicts (a) schematic illustration of a 10-channels microfluidic chip for Raman spectrum analysis, (b) photo of 10-channels microfluidic chip, a NdFeB permanent magnet (45×10×5 mm) was applied to collect Magchain to detection chamber, (c) microscopy imaging of detection chamber, (d) microscopy imaging of pneumatic valve at open and closed status, and (e) photo of Magchains collected in detection chamber (2 mins time lapse).

FIG. 17 depicts (a-b) TEM and (c) energy dispersive X-ray (EDX) elemental mapping analysis of SERS-encoded gold nanorods (AuNR) nanoprobes (SERS probes).

FIG. 18 depicts UV-Vis characterization analysis of SERS nanoprobes. Ultraviolet-visible (UV-Vis) spectrum of AuNR, AuNR@NTP@Ag, and AuNR@NTP@Ag@Streptavidin.

FIG. 19 depicts Raman spectra of AuNRs@NTP@Ag (SERS probe), AuNRs@NTP, NTP, and AuNRs.

FIG. 20 depicts SEM images of (a) control experiment without lysostaphin treatment, (b) control experiment without metabolic precursor, (c) Magchain—S. aureus—SERS probe sandwich complex. (scale bar: 1 μm).

FIG. 21 depicts capture rate of S. aureus (10³-10⁷ CFU/mL) by anti-S. aureus antibodies functionalized magnetic nanochains (Magchain) in Mueller Hinton Broth (MHB) pure culture and spiked sheep full blood.

FIG. 22 depicts (a) illustration of MILab-MiChip assay for rapid identification and susceptibility testing of S. aureus in a mice sepsis model, (b) standard colony counting of full blood samples post infection (3-48 h), (c) SERS responses in the analysis of blood samples before and post infection (6-24 h), and (d) MILab-MiChip used for rapid pathogen identification and antibiotics screening in different bacteria infected mice.

FIG. 23 depicts photographs of as-prepared nanochains (left) and PEGylated nanochains (right) at (a) 0 hour and (b) 2 hours in 180 mM phosphate buffered saline (PBS), and (c) shows electrophoresis of nanochains modified with different concentrations of aptamer (from 1 to 5, the concentrations are 0, 2.5, 5, 10, and 25 μg/mL). The method of manufacture of Magchain is hereby incorporated by patent U.S. Pat. No. 11,185,836B2.

### DESCRIPTION

In a first aspect of the invention, there is provided a method of detecting an antimicrobial susceptibility of a bacterium to one or more antibiotics, the method comprising the steps of:
(a) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor is labelled or is capable of being labelled with a detectable moiety; and
   a nutrient suspension; and
(b) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and:
   when the bacterial mixture of step (a) comprises a metabolic precursor that is capable of being labelled with a detectable moiety, then a detectable moiety suitable to conjugate to the metabolic precursor is added to the antibiotic-challenged bacterial mixture.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

It will be appreciated that the steps outlined in the method above are intended to occur in vitro as an analytical test.

It will also be appreciated that the steps of the method outlined above eventually enable one to assess whether a bacterial mixture presented for testing comprises a bacterium that is resistant to one or more antibiotics. This bacterial mixture may be obtained from a laboratory setting or from a subject suspected of having a bacterium that is resistant to one or more antibiotics. In situations where the subject is found to have a bacterial infection caused by a bacterium (or bacteria) that are resistant to one or more antibiotics, then the subject may be subjected to a method of medical treatment, wherein the method(s) outlined herein may further comprise the step of administering one or more antibiotics that the bacterium (or bacteria) are susceptible to, to the subject in order to treat the subject. As will be understood, by working out which antibiotic(s) the bacterium (or bacteria) are resistant and/or susceptible to, this will allow the skilled person to devise a suitable treatment for the subject.

When two or more antibiotics are administered to the subject, they may be administered sequentially, simultaneously or concomitantly. When used herein, the term “administered sequentially, simultaneously or concomitantly” includes references to:
administration of separate pharmaceutical formulations (one containing an antibiotic formulation (comprising one or more antibiotics) and one or more others containing one or more other antibiotics); and
administration of a single pharmaceutical formulation containing the two or more antibiotics.

The combination product described above provides for the administration of component (A) in conjunction with component (B), and may thus be presented either as separate formulations, wherein at least one of those formulations comprises component (A) and at least one comprises component (B), or may be presented (i.e. formulated) as a combined preparation (i.e. presented as a single formulation including component (A) and component (B)).

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

The bacterial species suspected to be resistant to one or more antibiotics may be any such bacterial species (or bacterium). Examples of suitable bacterial species types include, but are not limited to, methicillin-resistant Staphylococcus aureus (MRSA), vancomycin-resistant Enterococcus (VRE), carbapenem-resistant Enterobacteriaceae (CRE), carbapenem-resistant Klebsiella pneumoniae, multidrug-resistant strains of Staphylococcus aureus, Escherichia coli, Pseudomonas aeruginosa, Acinetobacter baumannii, Shigella, Salmonella Typhi, and Mycobacterium tuberculosis. Examples that may be mentioned in embodiments herein may be multidrug-resistant Staphylococcus aureus.

The term “antibiotic” may relate to the use of a single antibiotic compound for the purpose of determining antibiotic resistance to the antibiotic used. Alternatively, the term may refer to the use of two or more (e.g. 2, 3, 4) antibiotic compounds for the purpose of determining antibiotic resistance (or lack thereof) to the antibiotics used.

Any suitable antibiotic may be used herein. Examples include, but are not limited to, a penicillin (e.g. penicillin G), gentamycin, cefepime, vancomycin, tetracycline, erythromycin, norfloxacin, azithromycin, nitrofurantoin, neomycin, and combinations thereof. These antibiotics may be presented as the free base (or acid) or as a pharmaceutically acceptable salt or solvate thereof.

Pharmaceutically acceptable salts that may be mentioned include acid addition salts and base addition salts. Such salts may be formed by conventional means, for example by reaction of a free acid or a free base form of an antibiotic with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of an antibiotic in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+)-camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g.

D-glucuronic), glutamic (e.g. L-glutamic), a-oxoglutaric, glycolic, hippuric, hydrobromic, hydrochloric, hydriodic, isethionic, lactic (e.g. (+)-L-lactic and (+)-DL-lactic), lactobionic, maleic, malic (e.g. (−)-L-malic), malonic, (+)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

The antibiotic(s) mentioned herein may also be provided in the form of solvates of the antibiotic(s) and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

The method disclosed herein may further comprise the steps of:
(c) binding an antibody and/or an aptamer to a resistant bacterium, if present, in the antibiotic-challenged bacterial mixture; and
(d) detecting the presence or absence of the resistant bacterium.

As will be appreciated, the method disclosed herein also acts to detect a bacterium that is resistant to one or more antibiotics.

It will also be appreciated that the method disclosed herein may be run in parallel. This allows for a range of concentrations of each antibiotic (or antibiotic mixture) to be tested against the bacterial mixture that may include a bacterium that is resistant to one or more antibiotics. Additionally or alternatively, these parallel tests may enable one to test the bacterial mixture against a panel of antibiotics (and antibiotic mixtures). In other words, this method may be run in a combinatorial manner to produce a number of results in a speedy manner, enabling the detection of an antibiotic (or antibiotic combination) that can be used to kill the bacterium, as well as giving information on the antibiotic(s) that the bacterium is resistant to.

As will be appreciated, the antibody and/or aptamer may be used as a recognition agent for a specific bacterium. As such, the antibody and/or aptamer may be generated to specifically target a specific bacterium. This may be achieved by means known in the art.

In such embodiments, the binding of the antibody and/or the aptamer allows for the separation of the resistant bacterium, if present, from the antibiotic-challenged bacterial mixture. In additional or alternative embodiments, the method may further comprise a step of increasing a concentration of the resistant bacterium, if present, before conducting a detection step.

The term “antibody” when used herein may refer to an isolated antibody, a purified antibody, an antibody fragment (e.g. single-chain antibodies and nanobodies), a monoclonal antibody or combinations thereof.

An “isolated” or “purified” antibody is one which has been identified and separated or recovered, or both, from a component of its natural environment. Contaminant components of an isolated antibody's natural environment are materials that would interfere with diagnostic uses of the antibody. Non-limiting examples of such contaminants include enzymes, hormones, and other proteinaceous or non-proteinaceous solutes.

The term “monoclonal antibody” as used herein refers to a preparation of antibody molecules of single molecular composition. A monoclonal antibody displays a single binding specificity and affinity for a particular epitope. The monoclonal antibodies herein specifically include “chimeric” antibodies in which a portion of the heavy or light chain, or both, is identical with or homologous to corresponding sequences in antibodies derived from a particular species or belonging to a particular antibody class or subclass, while the remainder of the chain or chains are identical with or homologous to corresponding sequences in antibodies derived from another species or belonging to another antibody class or subclass, as well as fragments of such antibodies so long as they exhibit the desired biological activity.

The term “antibody fragment” refers to a portion of an intact antibody, wherein the portion preferably retains at least one, preferably most or all, of the functions normally associated with that portion when present in an intact antibody. Examples of antibody fragments include Fab, Fab′, F (ab′)2, and Fv fragments, linear antibodies, single-chain antibody molecules, and multispecific antibodies formed from antibody fragments.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding.

Aptamers are short sequences of artificial DNA or RNA that bind a specific target molecule.

In embodiments disclosed herein, the antibody and/or the aptamer may be an antibody.

The “metabolic precursor suitable for incorporation into a bacterium” refers to a compound that may be taken up by a bacterium and incorporated into it in some way, e.g. into the cell wall of the bacterium. As will be appreciated, as the bacterial mixture of step (a) comprises at least one antibiotic, only a bacterial species that is resistant to the antibiotic(s) will be able to grow and replicate. Thus, only a resistant bacterium will incorporate the metabolic precursor. More particularly, the metabolic precursors used herein are provided in two possible forms:
as a metabolic precursor that is labelled with a detectable moiety; or
as a metabolic precursor that is capable of being labelled with a detectable moiety.

In particular embodiments that may be mentioned herein, the metabolic precursor suitable for incorporation into a bacterium may be suitable for incorporation into a bacterial cell wall.

Examples of suitable metabolic precursors that are capable of being labelled with a detectable moiety which may be mentioned herein include, but are not limited to, lipid II, and, more particularly, a D-amino acid (e.g. D-alanine, D-lysine, D-phenylalanine, D-cysteine), 3-deoxy-D-manno-octulosonic acid (KDO), trehalose, fucose, and N-acetyl glucosamine. These compounds may be functionalized with a suitable moiety to enable their conjugation to a desired detectable moiety. For example, each of the lipid II, D-amino acid, 3-deoxy-D-manno-octulosonic acid (KDO), trehalose, fucose, and N-acetyl glucosamine may bear a click handle suitable to form one or more covalent bonds to a detectable moiety. Examples of suitable click handles may include, but are not limited to, azide, alkyne, trans-cycloctene (TCO), tetrazine, dibenzocyclooctyne (DBCO), and thiol. An example of a suitable metabolic precursor may be KDO-N₃ as discussed in the examples section below.

In such embodiments, the detectable moiety may then be conjugated to the metabolic precursor after the latter has been taken up and converted into a component of the bacterial cell (e.g. as part of the bacterial cell wall). The detectable moiety used herein may be directly detectable (e.g. by fluorescence) or may be indirectly detectable (e.g. through further downstream method steps that introduce further moieties that enable detection). Examples of suitable detectable moieties include, but are not limited to, a fluorescent dye, an enzyme, and a protein binding moiety (e.g. biotin). In embodiments where these are added to the bacterial mixture following the incorporation of the metabolic precursor into a component of the bacterial cell, then they may include a moiety that enables their conjugation to the metabolic precursor. For example, the fluorescent dye, enzyme, and protein binding moiety may bear a click handle (as discussed above) that complements that in the metabolic precursor taken up by the bacterial cell. An example of a suitable detectable moiety may be DBCO-biotin, as discussed in the examples section below.

As will be apparent from the above, the metabolic precursor that is labelled with a detectable moiety may be selected from the above metabolic precursors that have already been conjugated with the detectable moieties mentioned herein. This may be achieved using the same click-style chemistry discussed herein (and in the examples section), but before the metabolic precursor is provided to the bacterial mixture in step (a) above.

Any suitable method of detection may be used herein. For example, the method of detecting the presence or absence of the resistant bacterium may include, but is not limited to, fluorescent, colourimetric, surface-enhanced Raman spectroscopy (SERS), chemiluminescence, and electrochemical detection.

The method of detecting the presence or absence of the resistant bacterium may include, but is not limited to, a metabolic and immune co-labelling fluorescent assay, a microarray assay, an enzyme-linked immunosorbent assay (ELISA), a lateral flow assay, a chemiluminescence assay, an electrochemical assay, and a surface-enhanced Raman spectroscopy (SERS)-dependent microfluidic assay.

Particular embodiments of the methods discussed above will now be listed as embodiments of the invention.

Thus, in an embodiment of the invention, the method of detecting an antimicrobial susceptibility of a bacterium to one or more antibiotics may comprise the steps of:
(i) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor is labelled or is capable of being labelled with a detectable moiety; and
   a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
   when the bacterial mixture of step (i) comprises a metabolic precursor that is capable of being labelled with a detectable moiety, then a detectable moiety suitable to conjugate to the metabolic precursor is added to the antibiotic-challenged bacterial mixture prior to conducting step (iii);
(iii) placing a portion of the antibiotic-challenged bacterial mixture into a well comprising an antibody specific towards the bacterial species suspected to be resistant to one or more antibiotics to capture the bacterial species to provide a captured sample; and
(iv) providing a stimulus to the captured sample and detecting a signal in response to the stimulus, where a response indicates a bacterial species that is resistant to one or more antibiotics.

Thus, in a further embodiment of the invention, the method of detecting an antimicrobial susceptibility of a bacterium to one or more antibiotics may comprise the steps of:
(i) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor is labelled or is capable of being labelled with a detectable moiety; and
   a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
   when the bacterial mixture of step (i) comprises a metabolic precursor that is capable of being labelled with a detectable moiety, then a detectable moiety suitable to conjugate to the metabolic precursor is added to the antibiotic-challenged bacterial mixture prior to conducting step (iii);
(iii) contacting the antibiotic-challenged bacterial mixture with an antibody specific towards the bacterial species suspected to be resistant to one or more antibiotics to provide a bacteria-antibody conjugate mixture;
(iv) contacting the bacteria-antibody conjugate mixture with a labelled protein comprising a second detectable moiety that is capable of conjugating to the antibody to provide a metabolic-immune co-labelled conjugate mixture; and
(v) providing one or more stimuli to the metabolic-immune co-labelled conjugate mixture, where detection of a signal from both the first and second detectable moieties in response to the stimulus indicates a bacterial species that is resistant to one or more antibiotics.

It is noted that the metabolic labelling and the immune labelling do not interfere with each other (as demonstrated in the examples below). Without wishing to be bound by theory, it is believed that this lack of interference (between differing labelling functions) enables the methods disclosed herein to function.

In a further embodiment of the invention, the method of detecting an antimicrobial susceptibility of a bacterium to one or more antibiotics may comprise the steps of:
(i) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor comprises a protein binding moiety that can be detected or is capable of being conjugated to a protein binding moiety that can be detected; and
   a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
   when the bacterial mixture of step (i) comprises a metabolic precursor that is capable of being conjugated to a protein binding moiety, the protein binding moiety suitable to conjugate to the metabolic precursor is added to the antibiotic-challenged bacterial mixture prior to conducting step (iii);
(iii) breaking up the bacteria in the bacterial mixture with a suitable material to provide a bacterial fragment mixture;
(iv) placing a portion of the bacterial fragment mixture into a well comprising an antibody specific towards the bacterial species suspected to be resistant to one or more antibiotics to capture fragments of the bacterial species to provide a captured sample;
(v) providing a protein suitable to bind to the protein binding moiety to the captured sample, which protein is conjugated to an enzyme capable of producing a detectible signal when supplied with a suitable substrate, to provide a capture sample ready for detection; and
(vi) providing a suitable substrate to the capture sample ready for detection, where the presence of the substrate generates one or more of a chemiluminescence, an electrochemical and, more particularly, a colourimetric response, which response indicates a bacterial species that is resistant to one or more antibiotics.

In the above embodiment, the protein suitable to bind to the protein binding moiety may be streptavidin conjugated to horse radish peroxidase. Alternatively, the protein suitable to bind to the protein binding moiety may be streptavidin conjugated to alkaline phosphatase (AP).

The above method may be an ELISA method (enzyme-linked immunosorbent assay). While ELISA may be a colourimetric method, it may also function using chemiluminescence or an electrochemical response. Such responses may be induced using a suitable enzyme (e.g. a suitable horse radish peroxidase).

Thus, in an embodiment of the invention, the method of detecting an antimicrobial susceptibility of a bacterium to one or more antibiotics may comprise the steps of:
(i) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor comprises a protein binding moiety that can be detected or is capable of being conjugated to a protein binding moiety that can be detected; and
   a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
   when the bacterial mixture of step (i) comprises a metabolic precursor that is capable of being conjugated to a protein binding moiety, the protein binding moiety suitable to conjugate to the metabolic precursor is added to the antibiotic-challenged bacterial mixture prior to conducting step (iii);
(iii) breaking up the bacteria in the bacterial mixture with a suitable material to provide a bacterial fragment mixture;
(iv) providing a strip cassette comprising:
   a sample well comprising a conjugate pad and a sample pad in fluid communication with each other, the conjugate pad comprising a protein conjugated to a metal nanoparticle; and
   a membrane in fluid communication with the sample well, the membrane comprising a control line comprising an antibody suitable to capture a protein conjugated to a coloured microsphere or a metal nanoparticle and a test line comprising an antibody specific towards the bacterial species suspected to be resistant to one or more antibiotics to capture fragments of the bacterial species; and
(v) adding the bacterial fragment mixture to the sample well, where the bacterial fragment mixture mixes with the protein conjugated to a coloured microsphere or a metal nanoparticle in the conjugate pad to provide a coloured microsphere conjugate mixture or a metal nanoparticle conjugate mixture and the coloured microsphere conjugate mixture or the metal nanoparticle conjugate mixture passes through the test line and the control line, whereupon the visual detection of the control line and test line indicates a bacterial species that is resistant to one or more antibiotics.

Any suitable method to break up the bacteria may be used herein. Examples of suitable methods include, but are not limited to, enzymatic lysis, thermal lysis, and mechanical disruption to break up the bacteria. In the examples below, an antibacterial enzyme (Lysostaphin) which is capable of cleaving the crosslinking pentaglycine bridges found in the cell wall has been used, but this is only intended as an example and is not to be considered to be limiting.

The protein conjugated to the metal nanoparticle or coloured microsphere/particle may be streptavidin. The metal nanoparticles may be Au nanoparticles, Au nanorods or Au/Ag core/shell or alloy nanoparticles.

Thus, in an embodiment of the invention, the method of detecting an antimicrobial susceptibility of a bacterium to one or more antibiotics may comprise the steps of:
(i) providing a bacterial mixture comprising:
   a bacterial population including a bacterial species suspected to be resistant to one or more antibiotics;
   an antibiotic;
   a metabolic precursor suitable for incorporation into a bacterium, which metabolic precursor comprises a protein binding moiety that can be detected or is capable of being conjugated to a protein binding moiety that can be detected; and
   a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
   when the bacterial mixture of step (i) comprises a metabolic precursor that is capable of being conjugated to a protein binding moiety, the protein binding moiety suitable to conjugate to the metabolic precursor is added to the antibiotic-challenged bacterial mixture prior to conducting step (iii);
(iii) breaking up the bacteria in the bacterial mixture with a suitable material to provide a bacterial fragment mixture;
(iv) adding a plurality of magnetic chain structures to the bacterial fragment mixture to provide a Magchain bound bacterial fragment mixture, where the magnetic chain structures comprise a chain of magnetic particles having a polydopamine coating and an antibody specific towards the bacterial species suspected to be resistant to one or more antibiotics to capture fragments of the bacterial species;
(e) introducing the Magchain bound bacterial fragment mixture into an sample well of a microfluidic device and supplying a surface-enhanced Raman spectroscopy (SERS) probe bound to streptavidin to the sample well to form a magnetic chain-bacterial fragment-SERS probe sandwich complex in the sample well;
(f) manipulating the magnetic chain-bacterial fragment-SERS probe sandwich complexes through the microfluidic device to a concentration and detection well, followed by washing; and
(g) seeking to detect a Raman spectroscopic signal for the magnetic chain-bacterial fragment-SERS probe sandwich complex, where detection of the signal indicates a bacterial species that is resistant to one or more antibiotics.

The protein binding moiety may be biotin in the above embodiments that require a protein binding moiety.

Further aspects and embodiments will now be discussed by reference to the following non-limiting examples.

### EXAMPLES

### Reagents

3-Azido-D-alanine hydrochloride (D-Ala-N₃), 3-Azido-L-alanine hydrochloride (L-Ala-N₃), 6-Azido-D-lysine hydrochloride (D-Lys-N₃), 6-Azido-L-lysine hydrochloride (L-Lys-N₃), 4-Azido-D-phenylalanine (D-Phe-N₃) and 4-Azido-D-phenylalanine (L-Phe-N₃) were obtained from Baseclick GmbH (Munich, Germany). 8-Azido-3,8-dideoxy-D-manno-octulosonic acid (KDO-N₃), dibenzocyclooctyne (DBCO)-modified fluorescent dyes including DBCO-AF488, DBCO-Cy5, sulfo DBCO-PEG4-amine, and biotin-poly (ethylene glycol) (PEG) 4-N-hydroxysuccinimide (NHS) ester were purchased from Click Chemistry Tools (Scottsdale, US). Native streptavidin protein was bought from ProSpec-Tany TechnoGene Ltd (Rehovot, Israel). horseradish peroxidase (HRP)-conjugated streptavidin (streptavidin-HRP), anti-streptavidin antibody, 3,3′,5,5′-Tetramethylbenzidine (TMB) substrate, and enzyme-linked immunosorbent assay (ELISA) stop solution were obtained from Abcam (Cambridge, UK). Anti-S. aureus antibody and protein G-AF488 was bought from Thermo Fisher Scientific (Waltham, US). Antibiotics, 4′,6-diamidino-2-phenylindole (DAPI), and other chemicals were purchased from Sigma-Aldrich (St. Louis, US).

### Example 1

### Metabolic Labelling

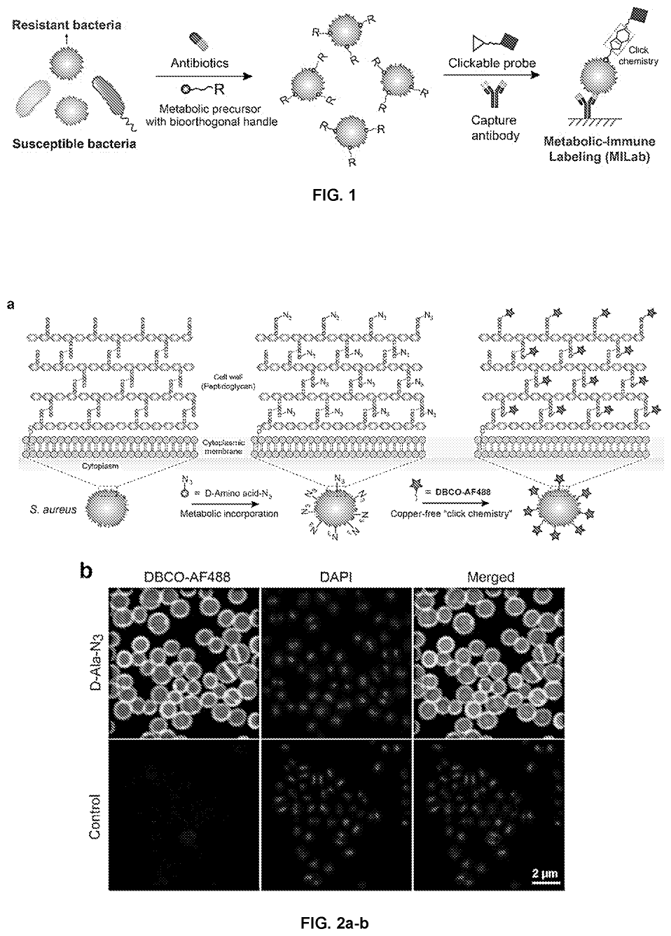

As illustrated in FIG. 1, the antibiotic-susceptible bacteria are inhibited or killed by the antibiotics, whereas the antibiotic-resistant bacteria remain metabolically active under antibiotics treatment. As a result, the metabolic precursors with a clickable azido group (e.g. D-amino acid-N₃, KDO-N₃) would selectively install click chemistry handles into the cell wall of resistant bacteria after antibiotic treatment, which could be subsequently labelled by DBCO-modified fluorescent dyes.

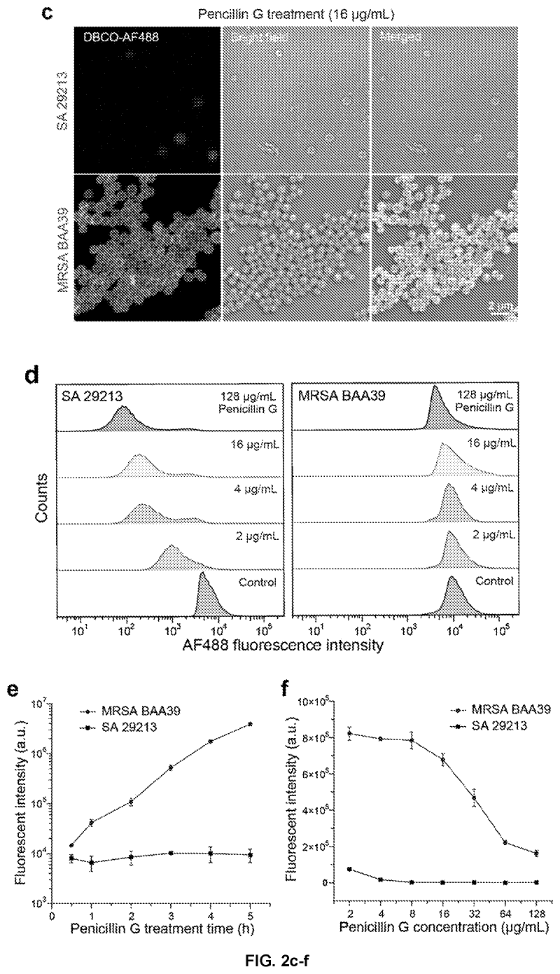

S. aureus is the leading cause of nosocomial bacteremia and the second leading cause of community acquired bacteremia. Here, S. aureus was chosen as a model Gram-positive bacterium to study the relationship between metabolic labelling and bacterial resistance. The mesh-like network of peptidoglycan (PG) is an essential constituent of bacterial cell walls. At the molecular level, PG contains repeating units of N-acetyl-glucosamine (NAG) linked N-acetyl-muramic acid (NAM), with the latter covalently linked to a pendant pentapeptide. As illustrated in FIG. 2a, the exogeneous D-amino acid analogues (e.g. D-Ala-N₃) will be incorporated into a newly synthesized PG of S. aureus and subsequently reacted with DBCO modified dyes (e.g. DBCO-AF488 and DBCO-Cy5) for fluorescence imaging in Example 2.

Moreover, we also studied the metabolic labelling of Gram-negative bacterium by using E. coli as a model.

### Bacterial Strains and Culture

We cultured the bacteria in nutrient-rich media (e.g. Luria-Bertani (LB) broth in the presence of D-Ala-N₃ of varying concentrations and durations. S. aureus (ATCC 29213), Multidrug-resistant S. aureus (BAA-38, BAA-39, and BAA-40), E. faecium (ATCC 51558), B. subtilis (ATCC 6633), S. mutans (ATCC 25175), E. coli (ATCC 8739), E. coli (EC 958), and C. difficile (ATCC 9689) were purchased from American Type Culture Collection (Manassas, US). S. aureus, E. faecium, B. subtilis, S. mutans and E. coli were grown in Brain-Heart Infusion (BHI) broth (Oxoid, UK) at 37° C. in a rotary shaker (180 rpm) and C. difficile was grown in Tryptic Soy Broth (TSB) supplemented with L-cysteine (0.1%) in an anaerobic chamber. The concentration of bacteria was determined by serial dilution with subsequent plating on agar plates and measurement of colony forming units (CFU).

Metabolic Labelling of Gram Positive and Negative Bacteria Overnight cultured bacteria were diluted 100 times in 1 mL of fresh medium containing 1.5 mM D-Ala-N₃ (Gram positive bacteria) or 3 mM KDO-N₃ (Gram negative bacteria) and incubated at 37° C. for 4 hours. The azido-labelled bacteria were then harvested by centrifugation at 4000 g for 5 min and washed two times with phosphate buffered saline (PBS, 0.01 M, pH 7.4).

The final pellet was resuspended in 0.2 mL PBS with 5 μM DBCO-AF488 or DBCO-Cy5 for click reaction. After 1 hour of click reaction at room temperature, the bacteria were collected by centrifugation and washed two times with 0.01 M PBS. Nuclear staining was performed with DAPI (10 μg/mL) for 10 min. The bacteria were finally fixed in 2% paraformaldehyde and washed two times with PBS prior to confocal microscopy and flow cytometry measurement.

### Confocal Microscopy

Labelled bacteria suspension (5 μL) was applied to a poly-lysine coated glass slide and a thin coverslip was then placed on it with slight pressure. Confocal microscopy was performed on a Zeiss LSM 800 or a Leica TCS SP8 confocal microscope. Samples were excited at 405 nm for DAPI, 488 nm for AF488, and 640 nm for Cy5, and the emission was detected using corresponding emission filters. Images were processed with ZEN 3.0 (Zeiss) or LAS X (Leica) software.

### Results and Discussions

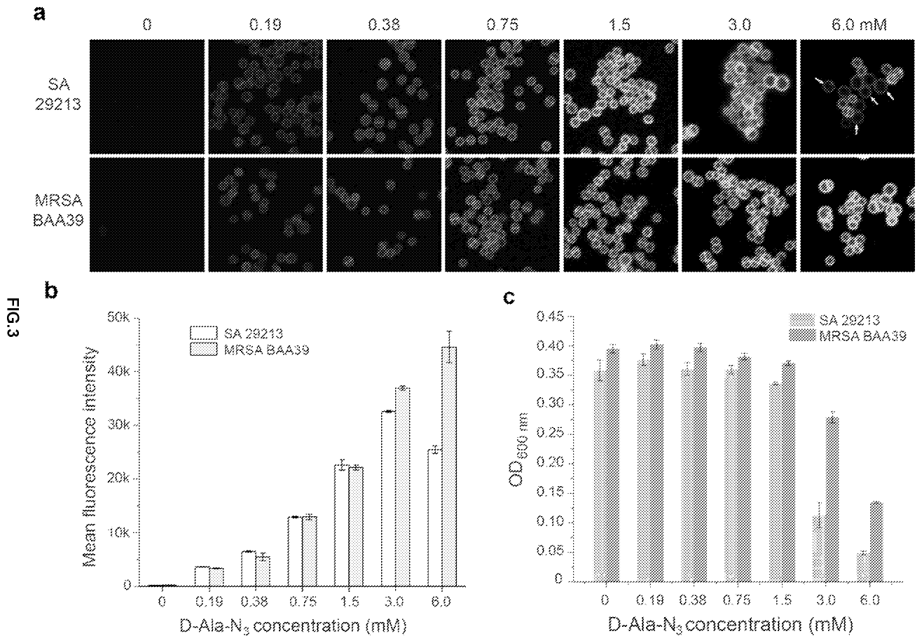

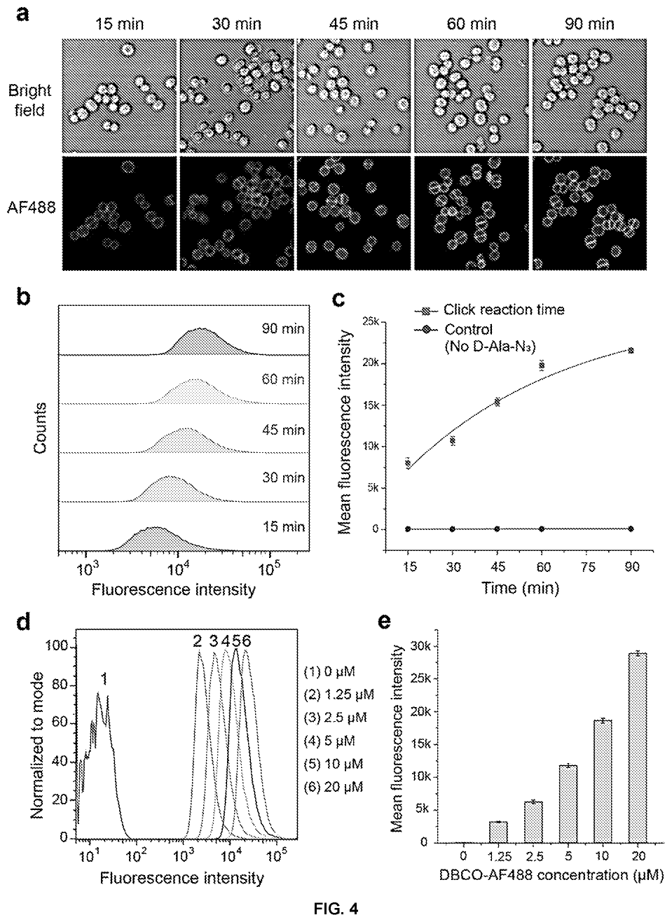

We found that the fluorescence intensity of labelling was dependent on the concentration of D-Ala-N₃ and the optimized concentration was 1.5 mM as higher concentration may inhibit the growth of bacteria S. aureus (Siegrist, M. S. et al., ACS Chem. Biol. 2013, 8, 500-505; Miyamoto, T. & Homma, H., J. Biochem. 2021; and Kolodkin-Gal, I. et al., Science 2010, 328, 627-629, FIG. 3). The catalyst-free click reaction was demonstrated very fast as the metabolic labelled bacteria could be clearly visualized at 15 min and quickly saturated at 1 h (FIG. 4a-c). It also demonstrated that the click reaction is in a concentration-dependent fashion. The fluorescence intensity of bacteria increased linearly with the DBCO-AF488 concentration without levelling off, suggesting that sufficient azido groups have been incorporated into bacterial cell wall (FIG. 4). FIG. 2b showed the metabolic labelling results for S. aureus under the optimized conditions. A blue DNA-stain dye DAPI was used to highlight the bacteria. It is very clear that the green clickable dye (e.g. DBCO-AF488) were intensively tagged on the bacterial envelops of S. aureus, suggesting that D-Ala-N₃ was successfully incorporated into the bacterial cell wall. In contrast, negligible labelling was found without the metabolic precursor D-Ala-N₃. It is noteworthy that strong metabolic labelling has appeared at the septum of the bacteria, where new PG components are freshly synthesized.

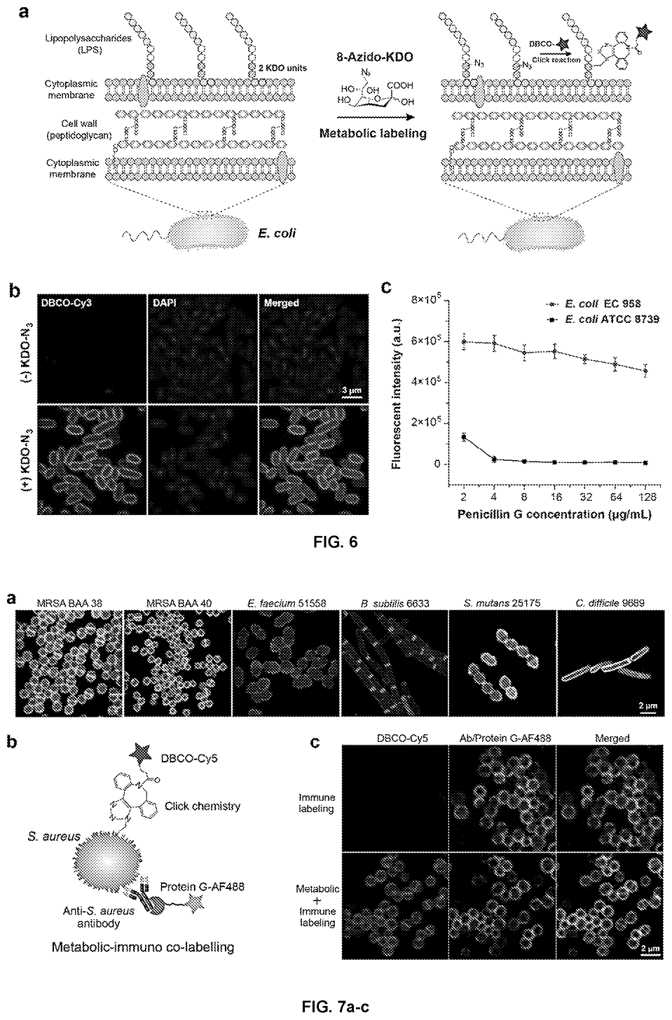

For E. coli metabolic labelling, the metabolic precursor KDO-N₃ at a concentration of 3 mM was added to the growth medium (LB broth), and the bacteria were growth for 4 hours. After stained by the clickable dye DBCO-Cy3, the bacteria showed a clear contour indicative of the stained lipopolysaccharides (LPS) on the outer membrane (FIG. 6).

### Example 2

### Bacterial Resistance at Varying Incubation Time

Due to the inhibition and killing effect of the antibiotics to the susceptible strains, antibiotic treatment afforded preferential labelling of drug-resistant bacteria, which remained metabolically active under antibiotics treatment. Hence, we are able to metabolically label specific resistant bacteria by tailored antibiotic selections.

### Preparation of Antibiotics Solution

The stock solutions of antibiotics were prepared according to the manufacturer's guidelines.

Penicillin G sodium, gentamicin sulfate salt, cefepime hydrochloride, vancomycin hydrochloride, and neomycin trisulfate salt were dissolved with deionized (DI) H₂O. Tetracycline, erythromycin, and azithromycin were dissolved with ethanol. Nitrofurantoin was dissolved in 1 mol/L NaOH solution and norfloxacin was dissolved in dimethylformamide (DMF). All the antibiotics were prepared at 10 mg/mL and preserved at −80° C. before usage.

Penicillin G at a concentration of 16 μg/mL was incubated with methicillin-susceptible S. aureus (SA 29213) and methicillin-resistant S. aureus (MRSA BAA39) together with D-Ala-N₃ for a certain time and then stained by DBCO-AF488 by following the protocol in Example 1.

### Results and Discussions

As shown in FIG. 2e, the metabolic labelling method could clearly distinguish susceptible and resistant S. aureus upon 0.5 h exposure of penicillin G. The fluorescence intensity of resistant bacteria increased quickly with increasing incubation time, whereas the fluorescence intensity of susceptible bacteria was kept constraint.

### Example 3

### Bacterial Resistance at Varying Concentrations

Varying concentrations of penicillin G were mixed with D-Ala-N₃ in the culture media to incubate with both bacteria by following the protocol in Example 2.

### Results and Discussions

It was found that penicillin G was able to completely inhibit the metabolic labelling of the susceptible bacteria. On the other hand, the resistant bacteria exhibited much stronger fluorescence intensity, although it slightly decreased with increasing concentration of penicillin G (FIG. 2f). The fluorescence intensity of bacterial suspension, however, mainly reflects the overall metabolic level (or growth level) rather than the metabolism rates of single bacteria. Hence, we used confocal imaging in Example 1 to observe the metabolic labelling of single bacteria under antibiotics treatment. The results confirmed that penicillin G treatment (16 μg/mL) led to selective labelling of resistant strains and negligible labelling of susceptible strains (FIG. 2c).

### Example 4

### Relationship Between Metabolism Individual Bacteria Between Antibiotics with Varying Concentrations

We used flow cytometry to further study the relationship between metabolism rates of individual bacteria and antibiotics with varying concentrations.

### Flow Cytometry

S. aureus 29213 and MRSA BAA39 (10⁵ CFU/mL) were incubated in Mueller Hinton Broth (MHB) containing 1.5 mM D-Ala-N₃ and penicillin G with varying concentration (0, 2, 4, 16, 128 μg/mL) for 3 h. Afterwards, all bacterial samples were washed twice and resuspended in 0.5 mL PBS. Flow cytometry was performed on a 5-Lasers Fortessa X20 (BD Biosciences) instrument in NTU Biosciences Research Centre. Samples were run at a low flow rate and 10,000 events were counted for each analysis with the forward-scatter (FSC) threshold setting at 1000. Data were analyzed and plotted by using FlowJo software (V 10.6.2).

### Results and Discussions

The results showed the resistant strains MRSA BAA 39 maintained significant labelling even after treatment by high concentration of antibiotics Penicillin G and the susceptible strains SA 29213 had considerably reduced labelling compared to the untreated controls (FIG. 2d). These results clearly confirm that the metabolic labelling is much lower in susceptible bacteria than in resistant bacteria under antibiotics treatment, suggesting that an antimicrobial susceptibility test is possible by quantifying the degree of metabolic labelling.

### Example 5

### Metabolic Immune Labelling (MILab)

To provide identification of the bacterial species, specific antibody against the bacteria was used to capture and identify the pathogen in the subsequent assays. The integration of metabolic and immune labelling (MILab) enables the simultaneous identification and susceptibility testing of AMR bacteria in a single step without subculture. The MILab technique is versatile and applicable to many well-established detection platforms such as microarray assay, enzyme-linked immunosorbent assay (ELISA) and lateral flow assay (LFA) for rapid and sensitive identification of multidrug-resistance S. aureus. Based on the measurement of metabolic labelling, a rapid susceptibility testing for S. aureus was established.

### Metabolic-Immune Labelling (MILab)

S. aureus and other Gram-positive bacteria were cultured in BHI broth containing 1.5 mM D-Ala-N₃ for 4 hours. The metabolic labelled bacteria were collected by centrifugation at 4000 g for 5 min. The harvested bacteria were fixed in 2% paraformaldehyde for 15 min and washed twice with PBS. Then, the bacteria were incubated with anti-S. aureus antibody (4 μg/mL) and DBCO-Cy5 (5 μM) for 1 hour at room temperature. The bacteria were subsequently spun down and washed twice with PBS. Protein G-AF488 (5 μg/mL) was applied for 30 min at room temperature. Finally, the co-stained bacteria were collected for confocal microscopy (as described in Example 1) and flow cytometry (as described in Example 4) analysis.

### Results and Discussions

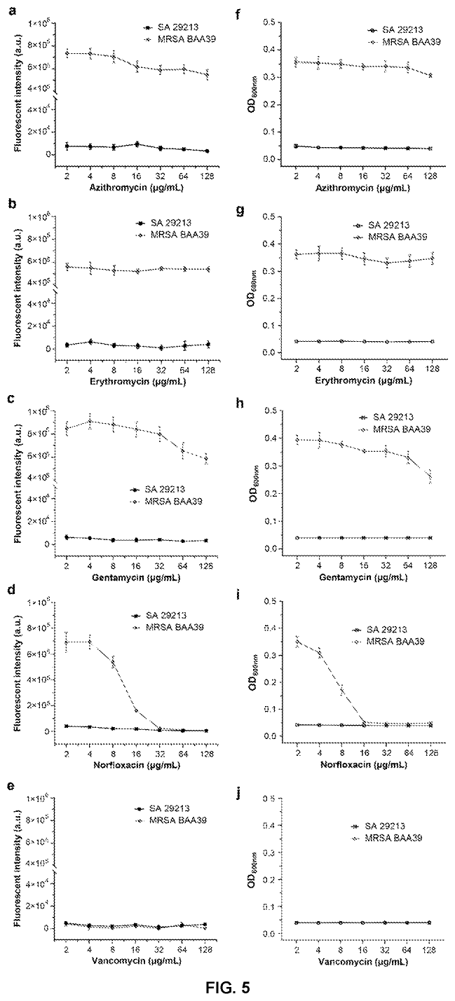

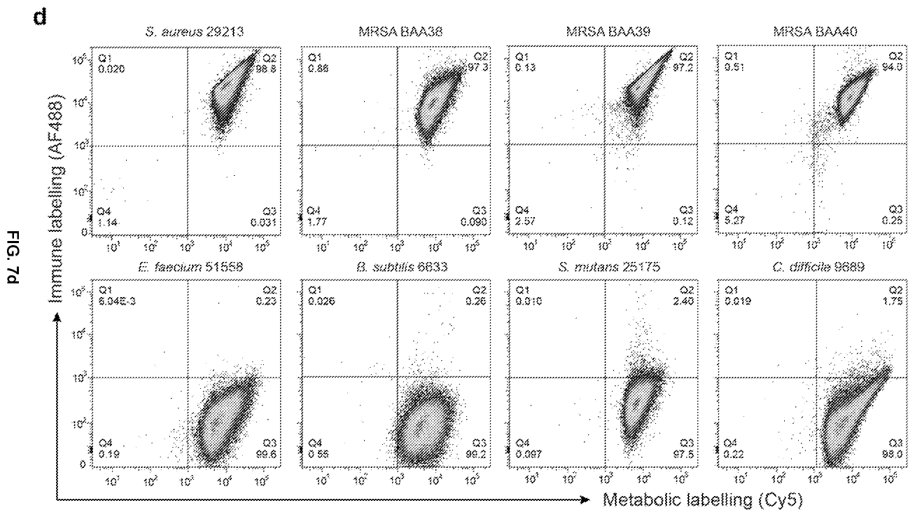

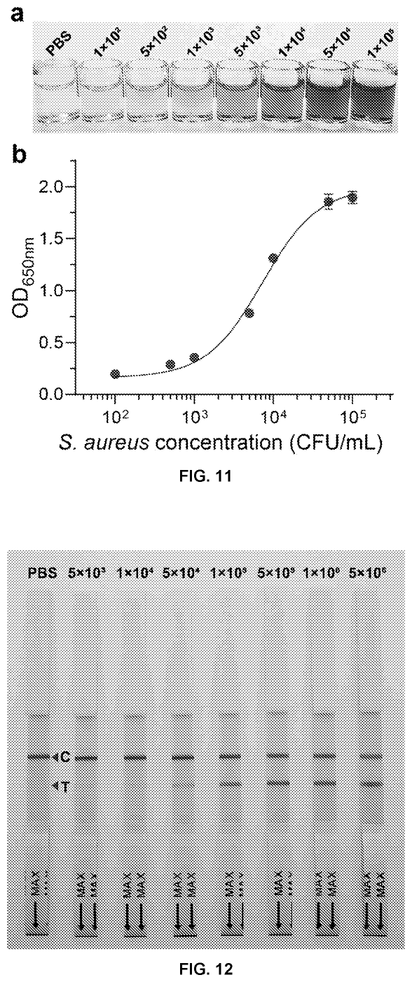

First, we verified that two MRSA (BAA38 and BAA40) and a panel of four Gram-positive bacterial species (Enterococcus faecalis, Bacillus subtilis, Streptococcus mutans and Clostridium difficile) have been noticeably labelled by DBCO-AF488, suggesting the metabolic precursor (D-Ala-N₃) was successfully incorporated into their cell wall (FIG. 7a). Next, a specific antibody against S. aureus was applied to capture and identify the pathogen in the subsequent assays. Here, it is crucial to demonstrate that the metabolic labelling will not interfere with antibody recognition. As illustrated in FIG. 7b, the azido group incorporated bacteria was simultaneously reacted with DBCO-Cy5 and anti-S. aureus antibody which was subsequently labelled by protein G-AF488. The confocal images showed the immune labelling intensity did not significantly change for the bacteria with or without metabolic labelling, hence demonstrating the metabolic labelling not interfering with immune labelling (FIG. 7c). Flow cytometry was used to measure the metabolic and immune labelling of a panel of four S. aureus and other four Gram-positive bacteria. The results showed that all four S. aureus strains were co-labelled and the other four Gram-positive bacteria were only metabolically labelled, suggesting that the antibody has excellent specificity for subsequent assays (FIG. 7d). The metabolic-immune labelling (MILab) method can be easily integrated into many well-established assay platforms (e.g. microarray assay, ELISA, lateral flow assay (LFA), microfluidic biochip) for rapid identification and susceptibility testing of drug-resistant S. aureus. The MILab based assays afford the detection and susceptibility testing of S. aureus with high sensitivity (100 CFU/mL) in a turnaround time of less than 5 hours (FIG. 11), which support antimicrobial stewardship and promote the prudent use of antibiotics. The robust metabolic labelling provides transduction signals for antimicrobial susceptibility testing (AST) and the specific immune recognition affords the bacterial identification, separation, and enrichment. It is important to demonstrate the metabolic labelling-based antimicrobial susceptibility testing (AST) is practicable for a panel of antibiotics with different treatment mechanism. The results of metabolic labelling-based AST are consistent with the results from standard broth dilution test (OD measurement). (FIG. 5)

### Example 6

### MILab Based Microarray Assay

In the past two decades, microarrays have been widely used in DNA, mRNA, protein and small molecules analysis. Microarray assays allow simultaneous detection of multiple analytes with low sample volume and reagent consumption, making it an ideal tool for high-throughput analysis in biological research and clinical diagnosis. Here, we developed MILab based microarray assay (MILab-Microarray assay) for rapid identification and susceptibility testing of drug-resistant S. aureus.

Microarray Slide Preparation The specific antibody against S. aureus was spotted on the epoxy-coated glass slide (Arrayit Co.) using uArrayer (LabNEXT) with a pin diameter of 100 μm. The slide was then incubated at 25° C. overnight in 30-40% humidity environment. Afterwards, the slide was assembled with ProPlate frame (16 wells, Grace Bio-Labs) to create multiple reaction wells. Each well was blocked with 2% bovine serum albumin (BSA) solution for 1 hour and washed with PBS containing 0.1% tween-20 (PBST) for two times.

### MILab-Microarray Assay

Bacterial samples were co-cultured with D-Ala-N₃ and an antibiotics (selected from Penicillin G, Gentamicin, Cefepime, Vancomycin, Tetracycline, Erythromycin, Norfloxacin, Azithromycin, Nitrofurantoin, and Neomycin) in 0.5 mL Mueller Hinton Broth (MHB) broth at 37° C. After 2 hours of incubation, the bacterial cultures were centrifuged at 4000 g for 5 min and fixed in 2% paraformaldehyde for 15 min. Then, the azido-labelled bacteria were collected by centrifugation at 4000 g for 5 min. The pellet was resuspended in PBS containing 5 μM DBCO-AF488 and quickly added in the microarray reaction wells for 1 hour at 37° C. The MILab-Microarray assay wells were washed three times with PBST. The slide was disassembled from the frame and imaged by a total internal reflection (TIRF) microscopy (ECLIPSE Ti2, Nikon). The fluorescence images were acquired at 200 ms exposure and analyzed by MetaVue software.

### Scanning Electron Microscopy (SEM)

The microarray slide was cut into small pieces (1×1 cm) by a diamond cutter. Prior to loading in scanning electron microscopy (SEM), the small pieces of microarray were coated with ultra-thin gold at 20 mA by sputter coating. SEM images were captured using a field-emission scanning electron microscope (JSM-6700F).

### Results and Discussions

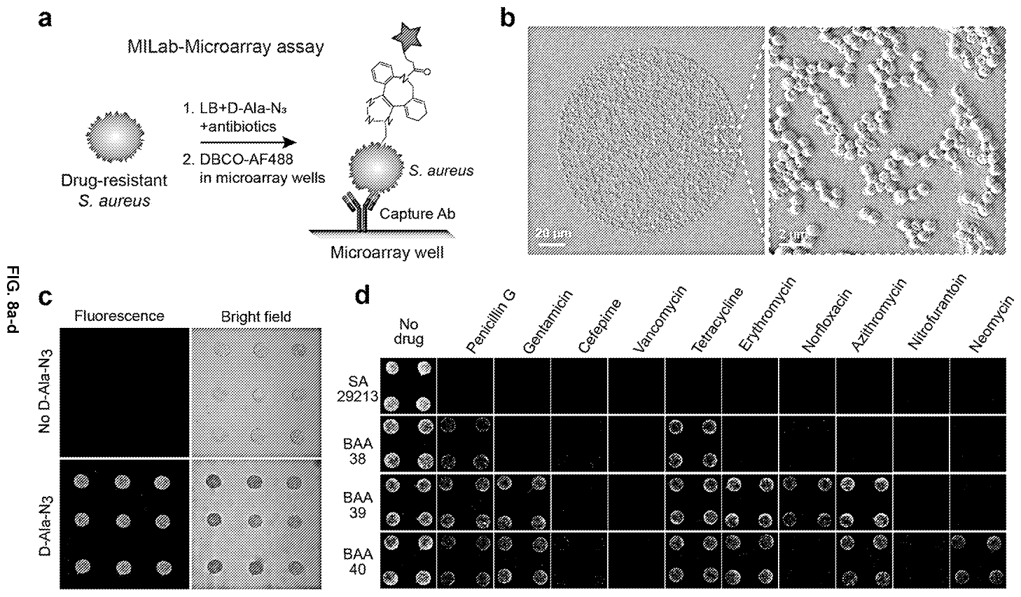

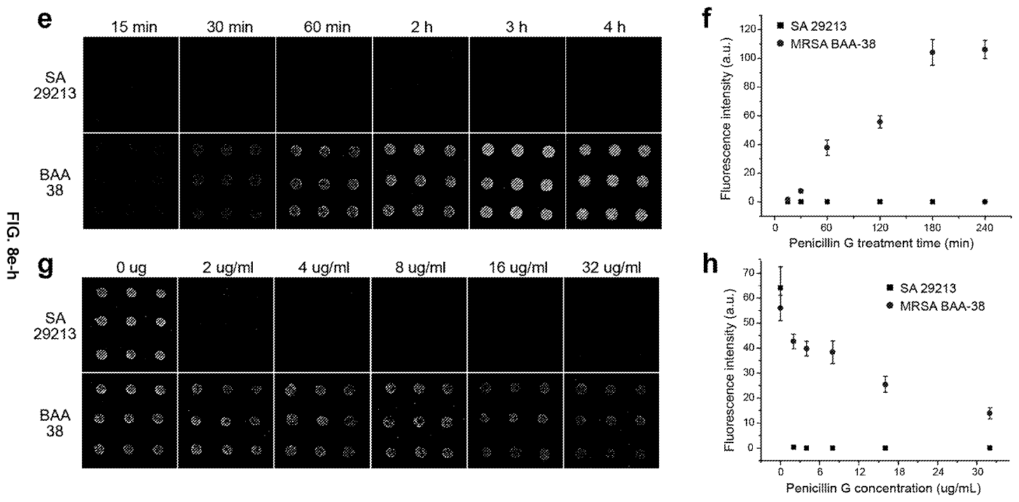

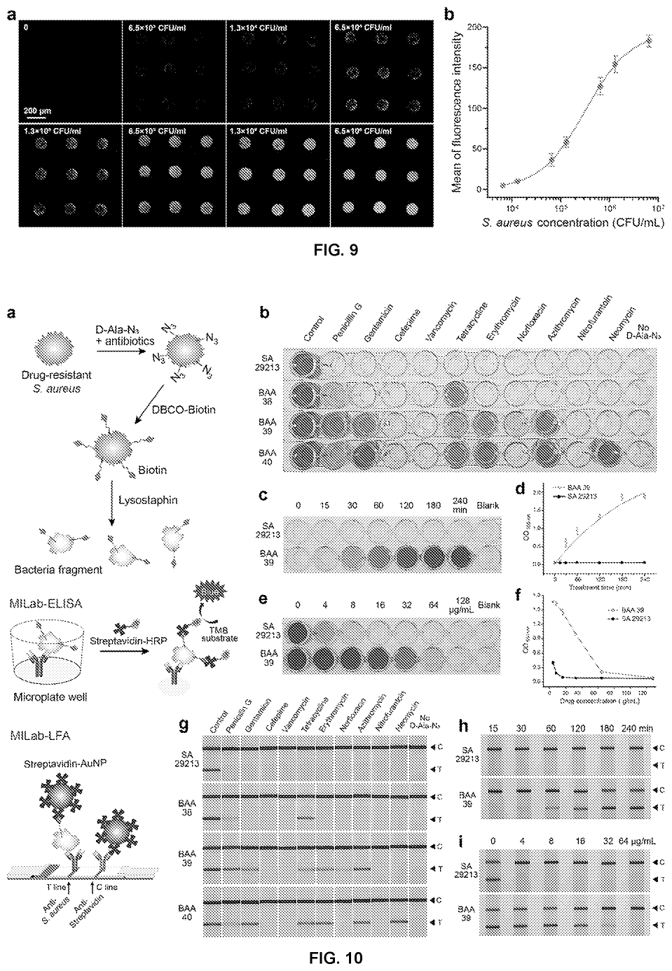

As shown in FIG. 8a, the specific antibody against S. aureus was immobilized on microarray substrates. In the assay, the immobilized antibody could specifically capture the metabolically labelled bacteria by immune recognition and meanwhile react with DBCO-AF488 by click reaction. The antigen-antibody reaction and click reaction occurred simultaneously, which reduced the assay turn-around time to within 4 hours. S. aureus with and without metabolic labelling were tested by the MILab-microarray assay. Scanning electron microscope (SEM) image showed the bacteria were captured on the microarray substrate by the immobilized antibodies (FIG. 8b). Fluorescence spots were clearly observed for S. aureus with metabolic labelling, while negligible fluorescence signal was detected for S. aureus without D-Ala-N₃ in the bacterial culture (FIG. 8c). Different concentration of S. aureus, ranging from 10³ to 10⁶ CFU/mL, were cultured in MHB with D-Ala-N₃ for 2 hours and followed by microarray assays. We demonstrated the fluorescence intensity is directly proportional to bacterial concentration and the limit of detection (LOD) is approximate 6.5×10³ CFU/mL (FIG. 9).

We further applied this MILab-microarray assay for susceptibility testing of S. aureus. One drug-susceptible S. aureus (SA 29213) and three MRSA (BAA 38, 39 and 40) were challenged by a panel of commonly used antibiotics (as described in Example 6) during bacterial culture and followed by the MILab-microarray assay. The high-throughput results help differentiating the drug-resistant bacteria and providing their resistance profile for the commonly used antibiotics (FIG. 8d). Furthermore, SA 29213 and MRSA BAA38 were tested by the MILab-microarray assay under different drug treatment time and drug concentration. As shown in FIG. 8e and FIG. 8f, the fluorescence signal of the MRSA BAA38 increased with increasing penicillin G treatment time (15 min to 4 hours) at the concentration of 16 μg/mL, whereas the drug-susceptible SA 29213 showed negligible signal even at 4 hours. Similarly, we treated the drug-susceptible and drug-resistant S. aureus by increasing drug concentrations (0 to 32 μg/mL) of Penicillin G for 2 hours. The drug-resistant S. aureus MRSA BAA38 maintained significant signal even after treatment by high concentration of penicillin G and susceptible strain SA 29213 showed considerably reduced signal compared to the untreated controls (FIG. 8g and FIG. 8h). The MILab-microarray assay provides the opportunities for screening a panel of antibiotics of interest for multiple specimens and examining key parameters such as antibiotic concentrations, treatment conditions or drug combinations.

### Example 7

### MILab Based ELISA

Enzyme-linked immunosorbent assay (ELISA) is the most commonly used tool in clinical diagnostics. However, ELISA has not been used for resistance analysis due to a lack of antibodies to discriminate drug-resistant bacteria. Here, the new type of MILab technique can be easily integrated with ELISA for susceptible testing and pathogen identification.

Synthesis of Water-Soluble Clickable Biotin Reagent (DBCO-Biotin) 10 mg of Sulfo DBCO-PEG4-Amine was dissolved in 1 mL DMF. 5 L of triethanolamine (TEA) was added to adjust the solution pH to 9.0. Then, 10.5 mg of biotin-PEG4-NHS ester was added quickly and kept stirring for 24 hours. Afterwards, the mixture was added dropwise into diethyl ether to precipitate the DBCO-PEG-biotin. The product was collected by centrifugation at 1000 g and washed twice with diethyl ether. The precipitate was dissolved in 1 mL DI H₂O, dialyzed against H₂O and lyophilized to yield colorless residue. Finally, the product was dissolved in DI H₂O at final concentration of 10 mM and preserved at −20° C. before usage.

### ELISA Kit Preparation

First, 100 μL of Anti-S. aureus antibodies (10 μg/mL in PBS) was added to microplate wells (Corning, US) and incubated at 37° C. for 1 hour. Then, all the wells were washed twice with PBST and blocked with 2.5% BSA solution at 37° C. for 1 hour. Finally, the microwells were washed twice with PBST and stored at 4° C. before usage.

### MILab-ELISA Assay

Bacterial samples were co-cultured with D-Ala-N₃ and antibiotics (as described in Example 6) (in 0.5 mL MHB) for 2 hours at 37° C. Then, the bacterial cultures were centrifuged at 4000 g for 5 min and washed twice with PBS. The pellets were resuspended in 0.5 mL PBS solution containing 100 μM of DBCO-biotin. Next, the mixtures were incubated for 1 hour at room temperature and subsequently centrifuged at 4000 g for 5 min. Afterwards, the pellets were resuspended in 0.2 mL PBS solution containing 1 μg/mL of lysostaphin. The mixtures were added quickly to anti-S. aureus coated microplate wells and incubated for 45 min at 37° C. Subsequently, the wells were washed three times with PBST, and 100 μL of streptavidin-HRP solution was added to the wells and incubate for 15 min at 37° C. The reaction wells were washed three times with PBST followed by 5 min incubation with 100 μL of TMB substrate.

Finally, all wells were supplemented with 650 nm stop solution (100 μL) and analyzed for absorbance at 650 nm.

### Results and Discussions

As illustrated in FIG. 10a, the drug resistant S. aureus first metabolically incorporated the D-Ala-N₃ into PG and subsequently reacted with DBCO-biotin, which provides the extendable reaction sites with streptavidin containing probes. Considering the pore size limitation of the mesh-like network of PG, the large molecular weight compounds (e.g. Streptavidin-HRP) may be hard to reach the inner reaction sites. Hence, we used lysostaphin to cleave the pentaglycine cross-bridges in between the stem peptides in the PG. Thus, more inner reaction sites were exposed on the bacteria fragments for biotin-streptavidin reaction. In the ELISA assay, the biotin tagged bacteria fragments were captured by the antibody coated on the microplate well and simultaneously reacted with Streptavidin-HRP to form sandwich complex, which oxidizes TMB substrates to produce blue color. As shown in FIG. 10b, a high-throughput screening of resistant S. aureus against a panel of antibiotics was realized by MILab-ELISA. The resistance profiles of four S. aureus strains were consistent with the results of MILab-Microarray assay. Importantly, the MILab-ELISA has extremely low detection limit at 100 CFU/mL, which is capable of detecting the relatively low concentrations of bacteria in blood of sepsis patients (FIG. 11). Next, SA 29213 and BAA39 were tested by the MILab-ELISA under different drug treatment time and drug concentration. The susceptible and resistant bacteria can be clearly differentiated after 30 min of drug treatment (FIG. 10c and FIG. 10d). At a concentration of 16 μg/mL, penicillin G was able to completely inhibit the metabolism of the susceptible bacteria (SA 29213), while the resistant bacteria (BAA39) exhibited high metabolic activity at this concentration (FIG. 10e and FIG. 10f).

### Example 8

### MILab Based LFA

Lateral flow assay (LFA) has attracted great interest due to their friendly user formats, short assay times, little interferences, low costs, and being easy to be operated by non-specialized personnel. Based on the MILab technique, we further developed MILab-LFA for rapid identification and susceptibility testing of drug-resistant S. aureus.

### LFA Strip Fabrication

Gold nanoparticles (AuNPs) with different diameters (15, 25, and 45 nm) were prepared using a modified trisodium citrate reduction method. 100 mL of 0.01% HAuCl₄ was heated to boiling in a flask briefly with vigorous stirring. Then, 1% trisodium citrate (1, 1.5, and 2 mL) was added quickly and the color of the solution changed from dark red to wine red color. The solution was kept boiling and stirring for another 10 min, then cooled to room temperature. The as-prepared AuNPs were characterized by UV-vis spectrometer.

The pH of the 10 mL AuNP solution was then adjusted to 6.0 by adding 0.1 M Na₂CO₃. Next, 150 μg of streptavidin protein was added into the AuNPs solution and the mixture was kept stirring for 1 hour. Then, 1 mL of 5% BSA was added and the mixture was kept stirring for 30 min. The streptavidin-AuNPs were collected by centrifugation at 6000 g for 10 min and resuspended in 1 mL conjugation buffer. The streptavidin-AuNPs solution was dispensed on conjugate pad at 4 μL/cm. As illustrated in FIG. 10a, the specific antibody against S. aureus and anti-streptavidin antibody were printed on the nitrocellulose (NC) membrane as the test and control line, respectively. After 2 hours drying in vacuum oven, the NC membrane, sample pad, conjugate pad, and absorption pad were assembled as the LFA test strips.

### MILab-LFA Assay

For MILab-LFA assay, the bacterial culture and treatment were the same as in ELISA in Example 7. Then, the biotin-labelled bacteria were lysed by incubation with lysostaphin (1 μg/mL) for 15 min at room temperature. Afterwards, 100 μL of bacterial samples were dropped into the sample well of strip cassettes. In the assay, the biotin-tagged bacteria fragments were captured by the anti-S. aureus at the test line and further reacted with streptavidin-conjugated gold nanoparticles (streptavidin-AuNP). The excess streptavidin-AuNP crossed the test line and was captured by the anti-streptavidin at the control line. The results of test lines were observed with the naked eye after 10 min.

### Results and Discussions

The results showed that MILab-LFA can also be used for rapid screening of resistant bacteria against a panel of antibiotics (as described in Example 6) (FIG. 10g). In comparison with ELISA, MILab-LFA has lower sensitivity (FIG. 12) and was limited to qualitative analysis. The same samples under different drug treatment time and drug concentration were tested on the MILab-LFA strips. The results showed that at 4 μg/mL and 64 μg/mL, penicillin G could completely inhibit the susceptible bacteria (SA 29213) and resistant bacteria (BAA39), respectively (FIG. 10h). The resistant bacteria (BAA39) showed signal after 30 min of bacterial incubation, while susceptible bacteria (SA 29213) had no signal even after 4 hours of bacterial incubation (FIG. 10i).

### Example 9

### MILab-Based Microfluidic Biochip Assay

Microfluidic technology has experienced explosive growth, especially in the development of miniaturized, low-cost, and point-of-care (POC) diagnostic platforms, due to its advantages over conventional benchtop assays such as low samples and reagents consumption, short reaction time and high compatibility with various detection tools. Here, MILab technique was combined with the magnetic nanochain integrated microfluidic biochip (MiChip) assay for rapid bacterial identification and AST. The MiChip assay was built upon two core components, namely bioconjugated magnetic nanochain (Magchain) and surface-enhanced Raman scattering (SERS) nanoprobe. Importantly, the use of Magchain integrates the key functions of microfluidic bioanalysis into a streamlined process, which considerably simplifies the assay and microfluidic system design. Herein, a model 10-channel chip that can be easily expanded further for a larger pool of samples was developed.

### Bioconjugated Magnetic Nanochain (Magchain)

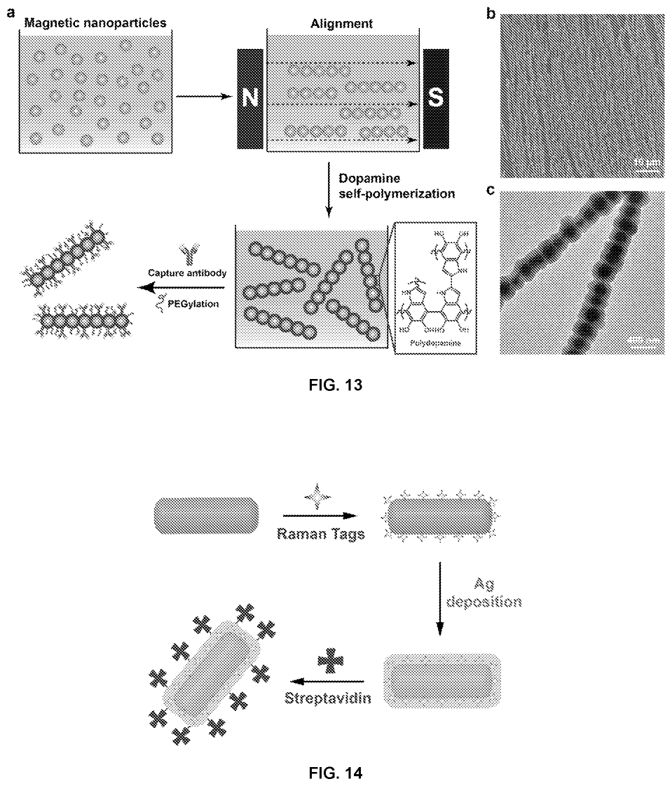

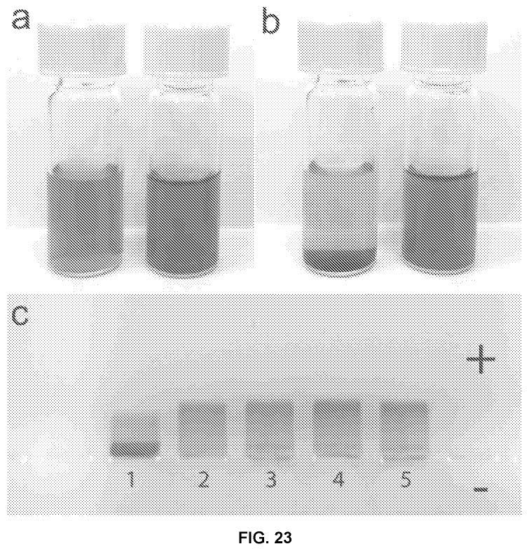

The method of manufacture of Magchain is hereby incorporated by patent U.S. Pat. No. 11,185,836B2.The Magchain was prepared by coating of polydopamine on the aligned magnetic nanoparticles in a homogeneous magnetic field and sequentially functionalized by bacteria-specific capture antibody and thiolated poly (ethylene glycol) (HS-PEG), which was introduced to suppress non-specific biofouling in the assay (FIG. 13 and FIG. 23). The bioconjugated Magchain can serve dual functions as nanoscale stir bar to promote rapid liquid mixing and as capture agent for specific separation of target bacteria on chip.

### Surface-Enhanced Raman Scattering (SERS) Nanoprobes

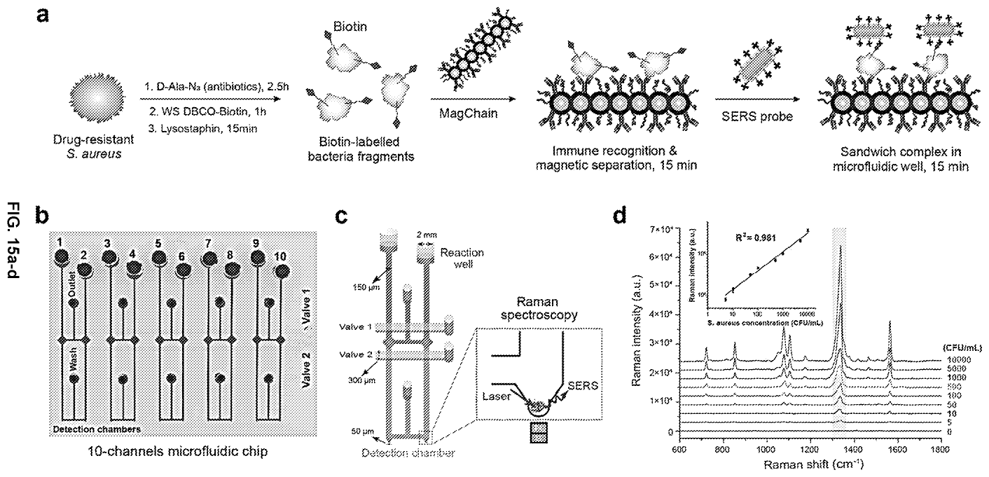

The SERS nanoprobes were prepared by anchoring Raman molecules such as 4-nitrothiophenol (4-NTP) on gold nanorods (AuNR) via the Au—S bond, then depositing a silver shell reduced by hydroquinone to embed the Raman molecules, and finally modifying streptavidin on the surface of core-shell Au@Ag nanorods (FIG. 14).

### MILab-based Microfluidic Biochip Assay

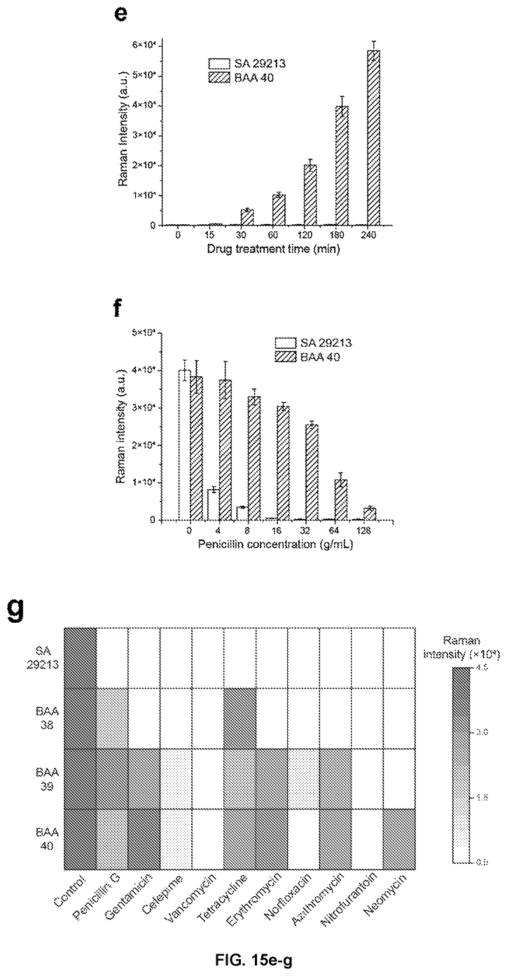

In the assay, the detection of drug-resistant bacteria was based on the formation of a sandwich structure by biotins-tagged bacteria, antibodies-conjugated Magchain, and streptavidin-functionalized SERS nanoprobes. As illustrated in FIG. 15a, azido groups was selectively installed on the drug-resistant S. aureus by metabolic labelling under antibiotic treatment and subsequently reacted with DBCO-biotin to introduce affinity tags on the bacteria. Upon lysostaphin treatment, the biotin-tagged bacterial fragments were recognized and separated by anti-S. aureus antibodies-conjugated Magchain and further reacted with SERS probes to form the sandwich complex.

MILab-Based 10-channels MiChip

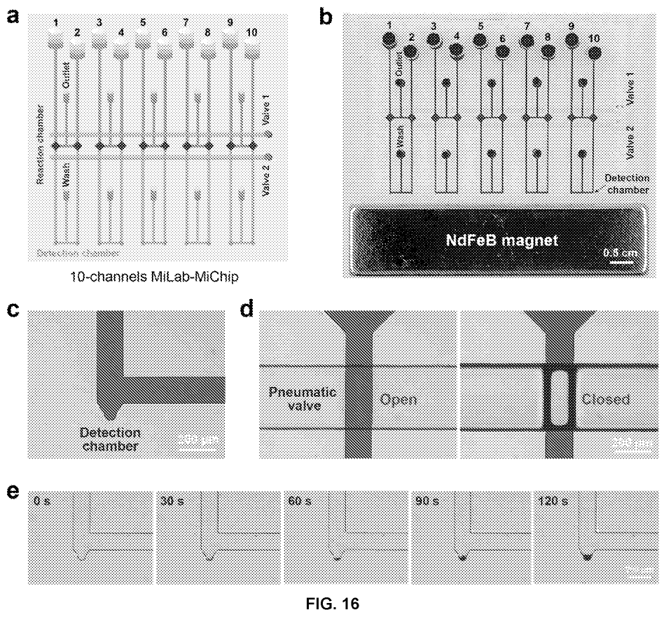

Herein, we developed a model 10-channels chip that can be easily expanded further for a larger pool of samples (FIG. 15b and FIG. 16). The basic unit of the MiChip platform consists of two reaction wells, two detection chambers, a washing inlet, a waste outlet, and two pneumatic microvalves that control the fluid delivery (FIG. 15c). Prior to the on-chip assay, the specimens containing biotin-tagged bacterial fragments were mixed with Magchains in a microwell plate for 15 min. The target bacterial fragments were captured by Magchains and enriched in a small aliquot (1/10 volume) by magnetic separation. The samples (10 μL) were then mixed with SERS nanoprobes and loaded into the reaction wells of 10-channel chip, and pneumatic valves 1 and 2 were closed to allow them to bind via the specific streptavidin-biotin interaction under continuous stirring. Afterwards, the valves 1 and 2 were opened and a permanent magnet (45×10×5 mm) was applied to collect all the Magchains including those in sandwich complex into the detection chamber for Raman spectroscopic detection (FIG. 15c and FIG. 16).

### Results and Discussions

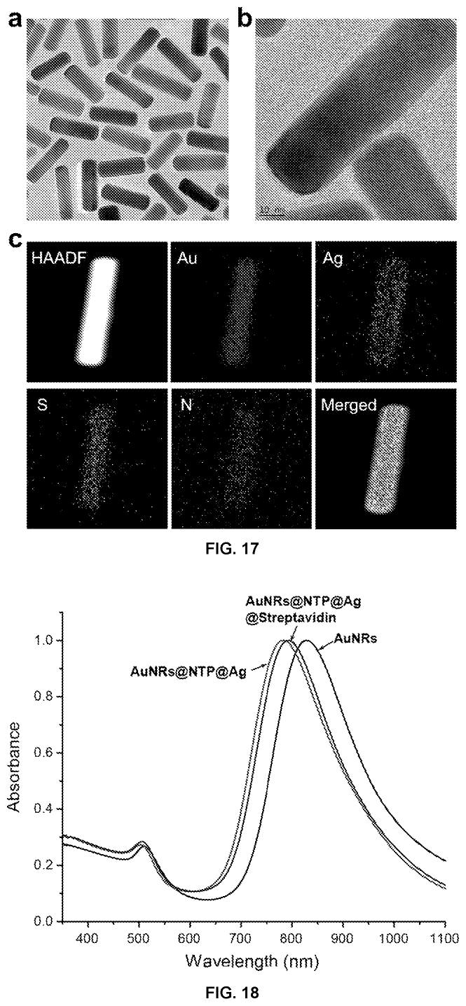

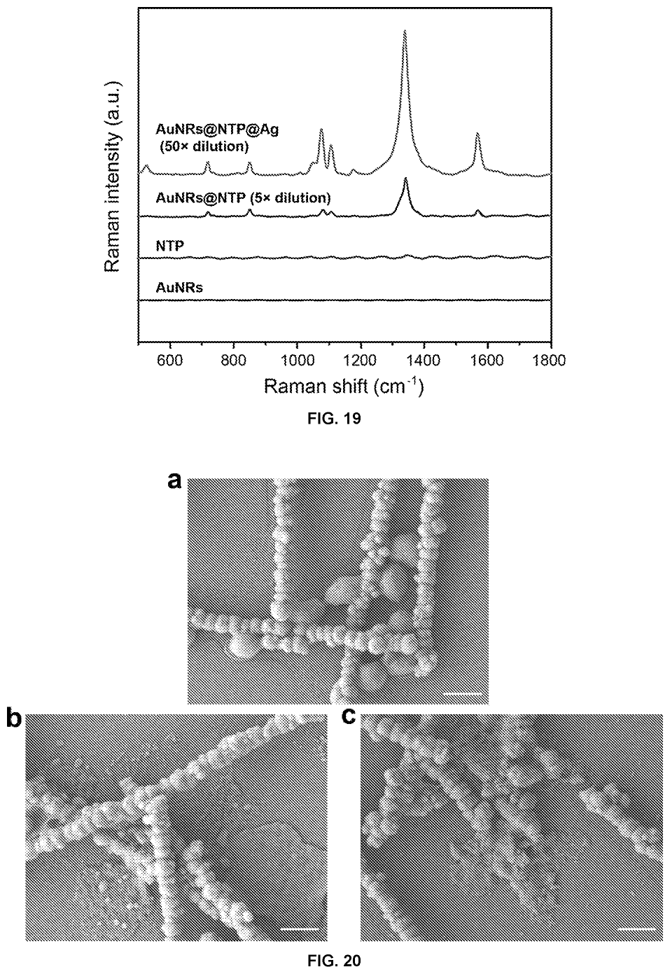

Transmission electron microscopy (TEM) imaging and energy dispersive X-ray (EDX) mapping analysis of SERS-encode AuNR nanoprobes (SERS nanoprobes) showed the high uniform core-shell nanostructure and provided the spatial distribution of elements, respectively (FIG. 17). The UV-Vis spectrum of SERS nanoprobes revealed that the AuNR were stable upon the deposition of Ag and functionalization of streptavidin (FIG. 18). The resultant SERS nanoprobes showed distinct longitudinal localized surface plasmon resonance (LSPR) centered at 790 nm, which was matched with Raman laser source (e.g. 785 nm) for efficient excitation. Remarkably, the 4-NTP-embedded core-shell SERS nanoprobes exhibited greatly amplified Raman signals, making them excellent for ultrasensitive detection (FIG. 19). Furthermore, the design of embedding Raman molecules inside the core-shell Au@Ag nanorods can effectively minimize the signal interference from external environment, which is critical for quantitative analysis.

Scanning electron microscope (SEM) images of Magchain—S. aureus—SERS probe sandwich complex showed the bacterial fragments were captured by Magchain and numerous SERS nanoprobes were attached on the fragments, whereas control experiments without metabolic substrates or lysostaphin treatment indicated no SERS nanoprobes attachment (FIG. 20).

### Example 10

### Sensitivity of MILab-Based MiChip Assay

To evaluate the sensitivity of MILab-based MiChip assay, a series of samples containing varying concentrations of S. aureus, ranging from 5 to 10⁴ CFU/mL, were conducted on the assay platform as described in Example 9. We measured the Raman signals and plotted the responses against the bacterial concentration in the samples.

### Results and Discussions

The Raman intensity at the characteristic peak (1341 cm⁻¹) showed consistent linear correlation (R²=0.981) with the bacterial concentration across a wide range (FIG. 15d). Notably, the MILab-based MiChip assay demonstrated extremely low detection limit at 5 CFU/mL, making it possible for detection of low concentrations of bacteria in clinical samples.

### Example 11

### Utility of MILab-Based MiChip Assay

We further evaluated the utility of this assay for examining key antibiotic-susceptibility information and screening a panel of antibiotics of interest for multiple specimens as described in Example 9.

Using MILab-based MiChip assay to distinguish a drug-susceptible (SA 29213) and a drug-resistant (BAA40) S. aureus

The antibiotic exposure time to distinguish a drug-susceptible (SA 29213) and a drug-resistant (BAA40) S. aureus by using the MILab-based MiChip assay was evaluated. The susceptible and resistant S. aureus (1×10⁴ CFU/mL) were cultured in MHB containing D-Ala-N₃ (1.5 mM) and penicillin G (16 μg/mL) for varying incubation time. The bacterial cultures were processed with DBCO-biotin, lysostaphin, and finally tested on the MiChip platform.

MILab-based MiChip platform for penicillin G dose-dependence susceptibility tests of a drug-susceptible (SA 29213) and a drug-resistant (BAA40) S. aureus

The use of the MILab-based MiChip platform for penicillin G dose-dependence susceptibility tests towards the two S. aureus strains was explored as described in Example 9.

10-Channels MiChip for high-throughput screening for antibiotics against multiple S. aureus strains

The 10-channels MiChip was further utilized for high-throughput screening for antibiotics against multiple S. aureus strains. The four S. aureus strains were co-cultured with D-Ala-N₃ and various antibiotics for 2.5 h, followed by MiChip testing.

### Results and Discussions

The resistant S. aureus BAA40 showed distinct Raman signal as short as 30 min of incubation time, while the susceptible S. aureus 25922 generated negligible signals all the time (FIG. 15e).

When using the MILab-based MiChip platform for penicillin G dose-dependence susceptibility tests towards the two S. aureus strains, the Raman intensity revealed their susceptibility profiles to the antibiotic (FIG. 15f). Notably, the susceptible and resistant S. aureus generated weak Raman signals upon exposure of penicillin G at 8 and 128 μg/mL, respectively. Thus, higher concentrations that are 2-4 times of minimal inhibitory concentration (MIC) should be applied to completely constrain or kill the strains.

When utilizing the 10-channels MiChip for high-throughput screening for antibiotics against multiple S. aureus strains, the Raman responses were plotted as heat map, which provides a guideline to choose the effect bacteria-antibiotics combination in treatment (FIG. 15g).

### Example 12

### Feasibility of Using Magchains to Separate S. aureus Spiked into Whole Sheep Blood

Prior to the processing of real bacteremia samples, we evaluated the feasibility of using Magchains to separate S. aureus that spiked into whole sheep blood at varying concentration as described in Example 9. The separation efficiency was calculated by enumeration of bacteria in the supernatant using standard colony counting method.

### Results and Discussions

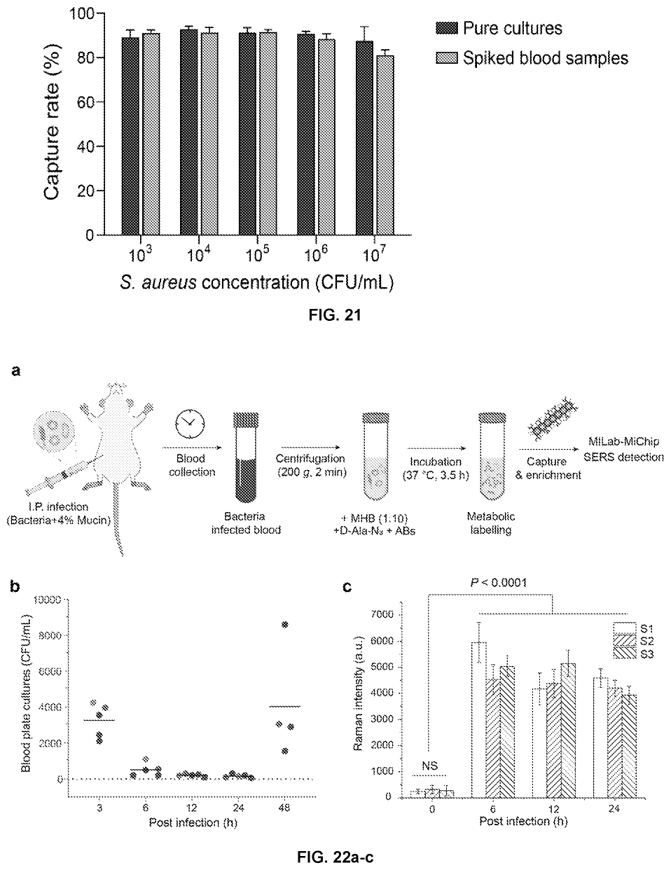

The results showed that the separation efficiencies of all concentration of S. aureus (10³-10⁷ CFU/mL) in full blood are more than 80%, which are comparable with the results for separation of pure culture S. aureus (FIG. 21).

### Example 13

### MILab-MiChip Application in Animal Sepsis Model

Sepsis is a serious clinical condition that can lead to multiple organ failure and has a high mortality rate of 28-50%. The emergence of multi-drug resistant bacteria makes the situation even more challenge in the treatment of sepsis. For patients with septic shock, the survival rates could increase as much as 9% for every hour that the effective antibiotic was administered. However, many patients with sepsis have extremely low bacterial concentration in blood (e.g. <100 CFU/mL), thus it is quite challenging for existing techniques to quickly identify the source of the infection and provide correct AST information directly from the clinical samples. To demonstrate the potential clinical utility of this MILab-based MiChip assay, we established an animal sepsis model by intraperitoneal (I.P.) infection. The whole blood was collected post infection in heparin lithium tube and further analyzed by the MILab-based MiChip platform.

### Intraperitoneal (I.P.) Infection

Overnight cultured bacteria were harvested by centrifugation (4000 g, 5 min) and suspended in sterile PBS containing 4% mucin to prepare the bacterial inoculum. Then, 0.2 mL of bacterial inoculum (10⁷ CFU/mL) was inoculated into the peritoneum of 7-week-old female BALC/C mice.

### Utility of MILab-Based MiChip Assay for Rapid Pathogen Identification and AST

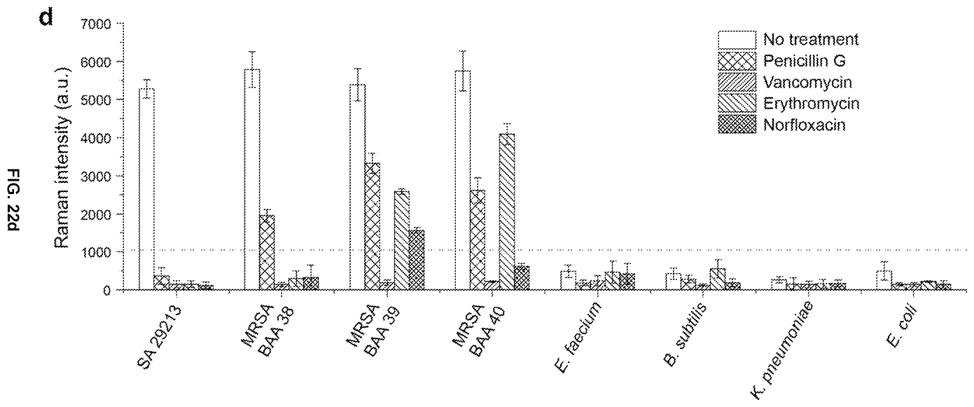

We further explored the utility of MILab-based MiChip assay for rapid pathogen identification and AST with real samples. As illustrated in FIG. 22a, the whole bloods from infected mice were first centrifuged at low speed (200 g, 2 min) to remove a large portion of blood cells. Then, the pre-treated blood samples were mixed with MHB at a 1:10 ratio and supplemented with D-Ala-N₃ (1.5 mM) and relevant antibiotic. Considering the low concentration and activity of bacteria in blood sample, we extended the incubation time to 3.5 h for efficient bacterial proliferation and metabolic labelling. Finally, the antibody conjugated Magchains were mixed with blood cultures on a stir plate for 15 min and magnetically transferred to reaction wells of microfluidic platform for MiChip assay.

### Results and Discussions

We first infected 5 mice with S. aureus and monitored the bacterial concentration in the mice blood by the standard colony counting method. The results showed that the bacterial concentrations in blood were quite low in 2 days post infection, especially the colony counts were less than 200 CFU/mL for most samples collected at 6-24 hours post infection (FIG. 22b). Thus, the current blood cultures for suspected sepsis often yielded false negative results due to the low bacterial concentration.

We next tested the blood samples collected before and 6-24 hours post infection by using this MILab-based MiChip assay. Significant Raman signals were always observed for testing blood samples post infection, demonstrating the high sensitivity of MILab-based MiChip assay (FIG. 22c).

Furthermore, we explored the use of MILab-based MiChip assay for high-throughput identification of pathogen and screening for antibiotics directly from full blood specimens. The blood specimens were collected from mice sepsis models through I.P. infection with various clinically relevant pathogens, including 4 strains of S. aureus, E. faecium, B. subtilis, K. pneumonia, and E. coli. We blindly tested these blood specimens following MILab-MiChip approach and measured the Raman intensity in response to a panel of antibiotics. The results showed this assay could specifically identity S. aureus (no treatment) and provide antibiotic-susceptibility information directly from full blood (FIG. 22d).

The entire assay procedure of bacterial identification and AST was completed within 6 h, which represents a major improvement from several days of current clinical practice. We envision that the high-throughput MILab-based MiChip assay will become a powerful tool for the management of bacteria-induced infectious diseases.

1. A method of detecting an antimicrobial susceptibility of a bacterium to one or more antibiotics, the method comprising the steps of:
(a) providing a bacterial mixture comprising:
a bacterial population including a bacterial species suspected to be resistant to one or more antibiotics;
an antibiotic;
a metabolic precursor suitable for incorporation into a bacterium, which metabolic precursor is labelled or is capable of being labelled with a detectable moiety; and
a nutrient suspension; and
(b) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and:
when the bacterial mixture of step (a) comprises a metabolic precursor that is capable of being labelled with a detectable moiety, then a detectable moiety suitable to conjugate to the metabolic precursor is added to the antibiotic-challenged bacterial mixture.

2. The method according to claim 1, wherein the method comprises further steps of:
(c) binding an antibody and/or an aptamer to a resistant bacterium, if present, in the antibiotic-challenged bacterial mixture; and
(d) detecting the presence or absence of the resistant bacterium.

3. The method according to claim 2, wherein the binding of the antibody and/or the aptamer allows for the separation of the resistant bacterium, if present, from the antibiotic-challenged bacterial mixture.

4. The method according to claim 2, further comprising a step of increasing a concentration of the resistant bacterium, if present, before conducting a detection step.

5. The method according to claim 1, wherein the metabolic precursor suitable for incorporation into a bacterium is incorporated into a bacterial cell wall.

6. The method according to claim 1, wherein the metabolic precursor suitable for incorporation into a bacterium is one that is capable of being labelled with a detectable moiety and is selected from one or more of the group consisting of lipid II, a D-amino acid, 3-deoxy-D-manno-octulosonic acid (KDO), trehalose, fucose, and N-acetyl glucosamine, where each of the lipid II, D-amino acid, 3-deoxy-D-manno-octulosonic acid (KDO), trehalose, fucose, and N-acetyl glucosamine bears a click handle suitable to form one or more covalent bonds to a detectable moiety.

7. The method according to claim 6, wherein the detectable moiety is selected from one or more of the group consisting of a fluorescent dye, an enzyme, and a protein binding moiety, wherein each of the fluorescent dye, enzyme, and protein binding moiety bears a click handle suitable to form one or more covalent bonds to the metabolic precursor suitable for incorporation into a bacterium that is capable of being labelled with a detectable moiety.

8. The method according to claim 6,
wherein the click handle on the metabolic precursor is selected from one or more of the group consisting of azide, alkyne, trans-cyclooctene (TCO), tetrazine, dibenzocyclooctyne (DBCO), and thiol;
the click handle on the metabolic precursor suitable for incorporation into a bacterium that is capable of being labelled with a detectable moiety complements the click handle on the detectable moiety.

9. The method according to claim 1, wherein the metabolic precursor suitable for incorporation into a bacterium that is labelled with a detectable moiety is selected from:
one or more of the group consisting of lipid II, a D-amino acid, 3-deoxy-D-manno-octulosonic acid (KDO), trehalose, fucose, and N-acetyl glucosamine; conjugated to one or more of the group consisting of a fluorescent dye, an enzyme, and a protein binding moiety.

10. The method according to claim 2, wherein the antibody and/or the aptamer is an antibody.

11. The method according to claim 2, wherein the step of detecting the presence or absence of the resistant bacterium involves one or more of fluorescent, colourimetric, surface-enhanced Raman spectroscopy (SERS), chemiluminescence, and electrochemical detection.

12. The method according to claim 2, wherein the method is a metabolic and immune co-labelling fluorescent assay, a microarray assay, an enzyme-linked immunosorbent assay (ELISA), a lateral flow assay, a chemiluminescence assay, an electrochemical assay, and a surface-enhanced Raman spectroscopy (SERS)-dependent microfluidic assay.

13. The method according to claim 2, wherein the method comprises the steps of:
(i) providing a bacterial mixture comprising:
a bacterial population including a bacterial species suspected to be resistant to one or more antibiotics;
an antibiotic;
a metabolic precursor suitable for incorporation into a bacterium, which metabolic precursor is labelled or is capable of being labelled with a detectable moiety; and
a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
when the bacterial mixture of step (i) comprises a metabolic precursor that is capable of being labelled with a detectable moiety, then a detectable moiety suitable to conjugate to the metabolic precursor is added to the antibiotic-challenged bacterial mixture prior to conducting step (iii);
(iii) placing a portion of the antibiotic-challenged bacterial mixture into a well comprising an antibody specific towards the bacterial species suspected to be resistant to one or more antibiotics to capture the bacterial species to provide a captured sample; and
(iv) providing a stimulus to the captured sample and detecting a signal in response to the stimulus, where a response indicates a bacterial species that is resistant to one or more antibiotics.

14. The method according to claim 2, wherein the method comprises the steps of:
(i) providing a bacterial mixture comprising:
a bacterial population including a bacterial species suspected to be resistant to one or more antibiotics;
an antibiotic;
a metabolic precursor suitable for incorporation into a bacterium, which metabolic precursor is labelled or is capable of being labelled with a detectable moiety; and
a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
when the bacterial mixture of step (i) comprises a metabolic precursor that is capable of being labelled with a detectable moiety, then a detectable moiety suitable to conjugate to the metabolic precursor is added to the antibiotic-challenged bacterial mixture prior to conducting step (iii);
(iii) contacting the antibiotic-challenged bacterial mixture with an antibody specific towards the bacterial species suspected to be resistant to one or more antibiotics to provide a bacteria-antibody conjugate mixture;
(iv) contacting the bacteria-antibody conjugate mixture with a labelled protein comprising a second detectable moiety that is capable of conjugating to the antibody to provide a metabolic-immune co-labelled conjugate mixture; and
(v) providing one or more stimuli to the metabolic-immune co-labelled conjugate mixture, where detection of a signal from both the first and second detectable moieties in response to the stimulus indicates a bacterial species that is resistant to one or more antibiotics.

15. The method according to claim 2, wherein the method comprises the steps of:
(i) providing a bacterial mixture comprising:
a bacterial population including a bacterial species suspected to be resistant to one or more antibiotics;
an antibiotic;
a metabolic precursor suitable for incorporation into a bacterium, which metabolic precursor comprises a protein binding moiety that can be detected or is capable of being conjugated to a protein binding moiety that can be detected; and
a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
when the bacterial mixture of step (i) comprises a metabolic precursor that is capable of being conjugated to a protein binding moiety, the protein binding moiety suitable to conjugate to the metabolic precursor is added to the antibiotic-challenged bacterial mixture prior to conducting step (iii);
(iii) breaking up the bacteria in the bacterial mixture with a suitable material to provide a bacterial fragment mixture;
(iv) placing a portion of the bacterial fragment mixture into a well comprising an antibody specific towards the bacterial species suspected to be resistant to one or more antibiotics to capture fragments of the bacterial species to provide a captured sample;
(v) providing a protein suitable to bind to the protein binding moiety to the captured sample, which protein is conjugated to an enzyme capable of producing a detectible signal when supplied with a suitable substrate, to provide a capture sample ready for detection; and
(vi) providing a suitable substrate to the capture sample ready for detection, where the presence of the substrate generates one or more of a chemiluminescence, an electrochemical and a colourimetric response, which response indicates a bacterial species that is resistant to one or more antibiotics.

16. The method according to claim 15, wherein the protein suitable to bind to the protein binding moiety is streptavidin conjugated to horse radish peroxidase.

17. The method according to claim 2, wherein the method comprises the steps of:
(i) providing a bacterial mixture comprising:
a bacterial population including a bacterial species suspected to be resistant to one or more antibiotics;
an antibiotic;
a metabolic precursor suitable for incorporation into a bacterium, which metabolic precursor comprises a protein binding moiety that can be detected or is capable of being conjugated to a protein binding moiety that can be detected; and
a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
when the bacterial mixture of step (i) comprises a metabolic precursor that is capable of being conjugated to a protein binding moiety, the protein binding moiety suitable to conjugate to the metabolic precursor is added to the antibiotic-challenged bacterial mixture prior to conducting step (iii);
(iii) breaking up the bacteria in the bacterial mixture with a suitable material to provide a bacterial fragment mixture;
(iv) providing a strip cassette comprising:
a sample well comprising a conjugate pad and a sample pad in fluid communication with each other, the conjugate pad comprising a protein conjugated to a metal nanoparticle; and
a membrane in fluid communication with the sample well, the membrane comprising a control line comprising an antibody suitable to capture a protein conjugated to a coloured microsphere or a metal nanoparticle and a test line comprising an antibody specific towards the bacterial species suspected to be resistant to one or more antibiotics to capture fragments of the bacterial species; and
(v) adding the bacterial fragment mixture to the sample well, where the bacterial fragment mixture mixes with the protein conjugated to a coloured microsphere or a metal nanoparticle in the conjugate pad to provide a coloured microsphere conjugate mixture or a metal nanoparticle conjugate mixture and the coloured microsphere conjugate mixture or the metal nanoparticle conjugate mixture passes through the test line and the control line, whereupon the visual detection of the control line and test line indicates a bacterial species that is resistant to one or more antibiotics.

18. The method according to claim 2, wherein the method comprises the steps of:
(i) providing a bacterial mixture comprising:
a bacterial population including a bacterial species suspected to be resistant to one or more antibiotics;
an antibiotic;
a metabolic precursor suitable for incorporation into a bacterium, which metabolic precursor comprises a protein binding moiety that can be detected or is capable of being conjugated to a protein binding moiety that can be detected; and
a nutrient suspension; and
(ii) aging the bacterial mixture for a period of time, then collecting the bacterial population and resuspending it to provide an antibiotic-challenged bacterial mixture, where a bacterium that is resistant to one or more antibiotics incorporates the metabolic precursor suitable for incorporation into the bacterium, and
when the bacterial mixture of step (i) comprises a metabolic precursor that is capable of being conjugated to a protein binding moiety, the protein binding moiety suitable to conjugate to the metabolic precursor is added to the antibiotic-challenged bacterial mixture prior to conducting step (iii);
(iii) breaking up the bacteria in the bacterial mixture with a suitable material to provide a bacterial fragment mixture;
(iv) adding a plurality of magnetic chain structures to the bacterial fragment mixture to provide a Magchain bound bacterial fragment mixture, where the magnetic chain structures comprise a chain of magnetic particles having a polydopamine coating and an antibody specific towards the bacterial species suspected to be resistant to one or more antibiotics to capture fragments of the bacterial species;
(v) introducing the Magchain bound bacterial fragment mixture into a sample well of a microfluidic device and supplying a surface-enhanced Raman spectroscopy (SERS) probe bound to streptavidin to the sample well to form a magnetic chain-bacterial fragment-SERS probe sandwich complex in the sample well;
(vi) manipulating the magnetic chain-bacterial fragment-SERS probe sandwich complexes through the microfluidic device to a concentration and detection well, followed by washing; and
(vii) seeking to detect a Raman spectroscopic signal for the magnetic chain-bacterial fragment-SERS probe sandwich complex, where detection of the signal indicates a bacterial species that is resistant to one or more antibiotics.

19. The method according to claim 15, wherein the protein binding moiety is biotin.

20. The method according to claim 7,
wherein the click handle on the detectable moiety is selected from one or more of the group consisting of azide, alkyne, trans-cyclooctene (TCO), tetrazine, dibenzocyclooctyne (DBCO), and thiol, provided that
the click handle on the metabolic precursor suitable for incorporation into a bacterium that is capable of being labelled with a detectable moiety complements the click handle on the detectable moiety.
